(19) United States
(12) Patent Application Publication
(10) Pub. No.: US 2024/0307517 A1
(43) Pub. Date: Sep. 19, 2024

(54) DEVELOPMENT OF A HIGHLY EFFICIENT SECOND GENERATION FENTANYL-CONJUGATE VACCINE TO TREAT FENTANYL ADDICTION

(71) Applicant: Cornell University, Ithaca, NY (US)

(72) Inventors: Ronald G. Crystal, New York, NY (US); Stephen Kaminsky, Bronx, NY (US); Tristan H. Lambert, Lansing, NY (US); Dolan Sondhi, New York, NY (US); Bishnu De, New Hyde Park, NY (US); Philip L. Leopold, Basking Ridge, NJ (US); Andrew Thomas Holm, Ithaca, NY (US)

(73) Assignee: Cornell University, Ithaca, NY (US)

(21) Appl. No.: 18/560,241

(22) PCT Filed: May 10, 2022
(86) PCT No.: PCT/US2022/028621

§ 371 Date: Nov. 10, 2023

Related U.S. Application Data
(60) Provisional application No. 63/186,451, filed on May 10, 2021.

(51) Int. Cl. A61K 39/00; A61K 39/385; A61K 47/64; A61P 37/04; C07D 211/58; C07D 401/04; C07D 471/04

(52) U.S. Cl. CPC A61K 39/0013; A61K 39/385; A61K 47/646; A61P 37/04; C07D 211/58; C07D 401/04; C07D 471/04; A61K 2039/6075; C12N 2750/14134

(57) ABSTRACT

The invention is directed to fentanyl analogues and a conjugate comprising same, as well as a method of inducing an immune response against fentanyl.

### CROSS-REFERENCE TO A RELATED APPLICATION

This patent application claims the benefit of U.S. Provisional Patent Application No. 63/186,451, filed May 10, 2021, the entire disclosure of which is hereby incorporated by reference.

### BACKGROUND OF THE INVENTION

Fentanyl is a highly addictive synthetic opioid that is used recreationally as a variant of heroin. Because fentanyl is highly potent (50-100-fold more than morphine), overdose fatalaties continue to rise. Vaccination is a proposed strategy for treating or preventing addiction and reducing the chance of a fatal overdose. However, as a small molecule, fentanyl is poorly immunogenic. Thus, there is a need for alternative compounds and compositions that can evoke an immunogenic response to fentanyl and be used to prevent or treat fentanyl addiction.

### BRIEF SUMMARY OF THE INVENTION

Provided herein are fentanyl analogues, and conjugates comprising same. Also provided herein are methods of using such analogues and conjugates to elicit an immunogenic response to fentanyl in a subject, and/or prevent or treat fentanyl addiction and/or reduce the likelihood of overdose, particularly fatal overdose, in a subject.

### BRIEF DESCRIPTION OF THE SEVERAL VIEWS OF THE DRAWINGS

FIG. 1 is a chemical scheme of the preparation of 6-(4-(2-(4-(2-oxo-3,4-dihydroquinolin-1(2H)-yl)piperidin-1-yl)ethyl)phenyl)hexanoic acid (analogue 4). Boc is tert-butoxycarbonly, DMAP is 4-dimethylaminopyridine, DIPEA is diisopropylethylamine, EDC is N-(3-dimethylaminopropyl)-N′-ethylcarbodiimide hydrochloride, and Ac is acetyl.

FIG. 2 is a chemical scheme of the preparation of 6-(2-oxo-1-(1-phenethylpiperidin-4-yl)-1,2,3,4-tetrahydroquinolin-6-yl)hexanoic acid (analogue 5). Boc is tert-butoxycarbonyl; Ac is acetyl DIPEA is diisopropylethylamine; Et is ethyl; Ph is phenyl.

FIG. 3 is a chemical scheme of the preparation of 6-(2-oxo-1-(1-phenethylpiperidin-4-yl)-1,2,3,4-tetrahydroquinolin-3-yl)hexanoic acid (analogue 6). DMSO is dimethyl sulfoxide.

FIG. 4 is a chemical scheme of the preparation of 6-(4-(N-(1,3,4,6,7,11b-hexahydro-2H-pyrido[2,1-a]isoquinolin-2-yl)propionamido)phenyl)hexanoic acid (analogue 7). TMS is trimethylsilyl, OTf is trifluoromethanesulfonate, TPAP is tetrapropylammonium perruthenate, NMO is N-methyl morpholine N-oxide, and THE is tetrahydrofuran.

FIG. 5 is a chemical scheme of the preparation of 7-((1,3,4,6,7,11b-hexahydro-2H-pyrido[2,1-a]isoquinolin-2-yl)(phenyl)amino)-7-oxoheptanoic acid (analogue 8).

FIG. 6 is a chemical scheme of the preparation of 6-(4-(2-(2-oxo-1-phenyloctahydro-1,6-naphthyridin-6(2H)-yl)ethyl)phenyl)hexanoic acid (analogue 9).

FIG. 7 is a chemical scheme of the preparation of 6-(4-(2-oxo-6-phenethyloctahydro-1,6-naphthyridin-1(2H)-yl)phenyl)hexanoic acid (analogue 10).

FIG. 8A and FIG. 8B provide alternative chemical schemes of the preparation of 5-(2-oxo-6-phenethyl-1-phenyldecahydro-1,6-naphthyridin-3-yl)pentanoic acid (analogue 11).

FIG. 9 is a graph of serum antibody titers.

FIG. 10 is a graph of the results of a competive binding assay.

FIG. 11 is a graph of the results of a competitive binding assay.

FIG. 12A and FIG. 12B are graphs of circulating fentanyl in the brain (FIG. 12A) and serum (FIG. 12B).

FIG. 13 is a graph of the results of an antinociception assay.

FIG. 14A and FIG. 14B are graphs of the results of a fentanyl-induced conditioned place preference assay.

FIG. 15A and FIG. 15B are graphs of serum antibody titers.

FIG. 16A and FIG. 16B are graphs of the results of competitive binding assays.

FIG. 17A and FIG. 17B are graphs of the results of an antinociception assay.

FIG. 18 is a graph of the results of a locomotion assay showing distance travelled in various treatment and control groups.

FIG. 19 is a chemical scheme of the preparation of 6-(4-(N-(1-phenethylpiperidin-4-yl)propionamido)phenyl)hexanoic acid (Analogue 2).

FIG. 20 is a chemical scheme of the preparation of 6-oxo-6-((1-phenethylpiperidin-4-yl)(phenyl)amino)hexanoic acid (Analogue 3).

### DETAILED DESCRIPTION OF THE INVENTION

Provided herein is a fentanyl analogue having the structure of any of Formulae (1)-(7):

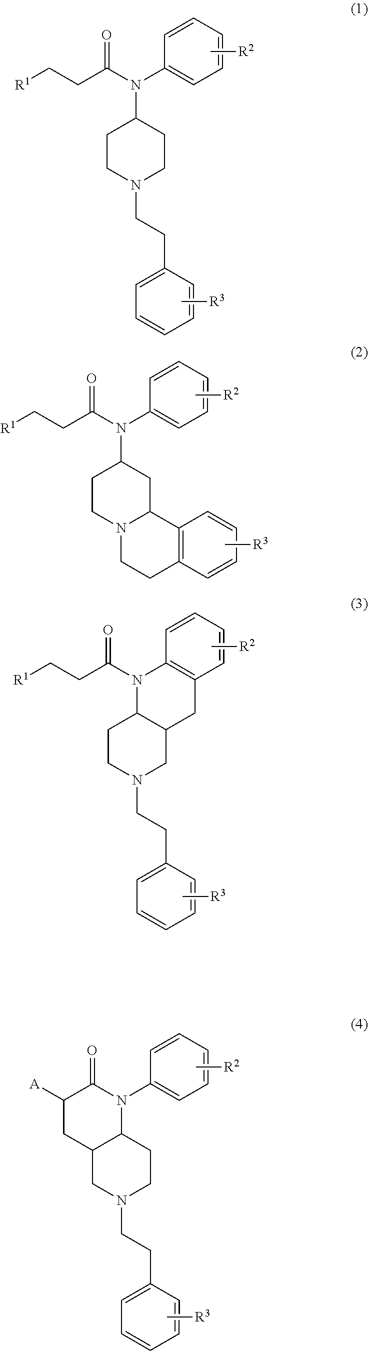

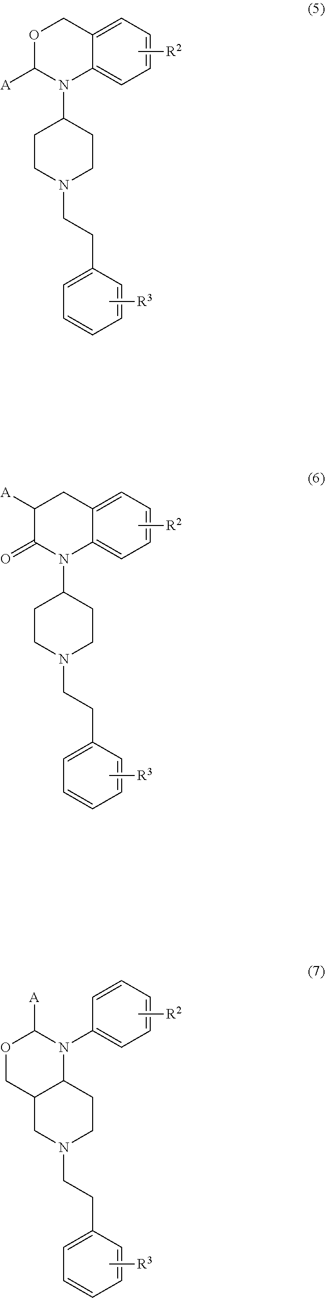

wherein A is H or -alkyl-R¹ and R¹, R², and R³ are each independently H or —(C₁-C₁₂ alkyl)-COOH, provided that at least one of R¹, R², and R³ is —(C₁-C₁₂ alkyl)-COOH. In some aspects of Formulae (1)-(7), only one of R¹, R², and R³ is —(C₁-C₁₂ alkyl)-COOH.

In some aspects of Formulae (1)-(7), A is H. In certain aspects, A is H and only one of R¹, R², and R³ is —(C₁-C₁₂ alkyl)-COOH

In some aspects of Formulae (1)-(7), A is -alkyl-R¹ (e.g., C₁₋₄ alkyl, C₁₋₂ alkyl, C₁-alkyl, or C₂-alkyl), R¹ and R² are H, and R³ is —(C₁-C₁₂ alkyl)-COOH. In other aspects of Formulae (1)-(7), A is -alkyl-R¹ (e.g., C₁₋₄ alkyl, C₁₋₂ alkyl, C₁-alkyl, or C₂-alkyl), R¹ and R³ are H, and R² is —(C₁-C₁₂ alkyl)-COOH. In still other aspects of Formulae (1)-(7), A is -alkyl-R¹ (e.g., C₁₋₄ alkyl, C₁₋₂ alkyl, C₁-alkyl, or C₂-alkyl), R² and R³ are H, and R¹ is —(C₁-C₁₂ alkyl)-COOH.

In any of the foregoing aspects, the alkyl portion of —(C₁-C₁₂ alkyl)-COOH can be a C₂-C₁₂ alkyl or C₃-C₁₂ alkyl, such as a C₂-C₈ alkyl or C₃-C₈ alkyl (e.g., a C₂, C₃, C₄, C₅, C₆, C— or C₈ alkyl). Preferably, the alkyl portion of —(C₁-C₁₂ alkyl)-COOH is C₅.

In some aspects, Formulae (1)-(7) are of the structure of Formulae (1′)-(7′), respectively:

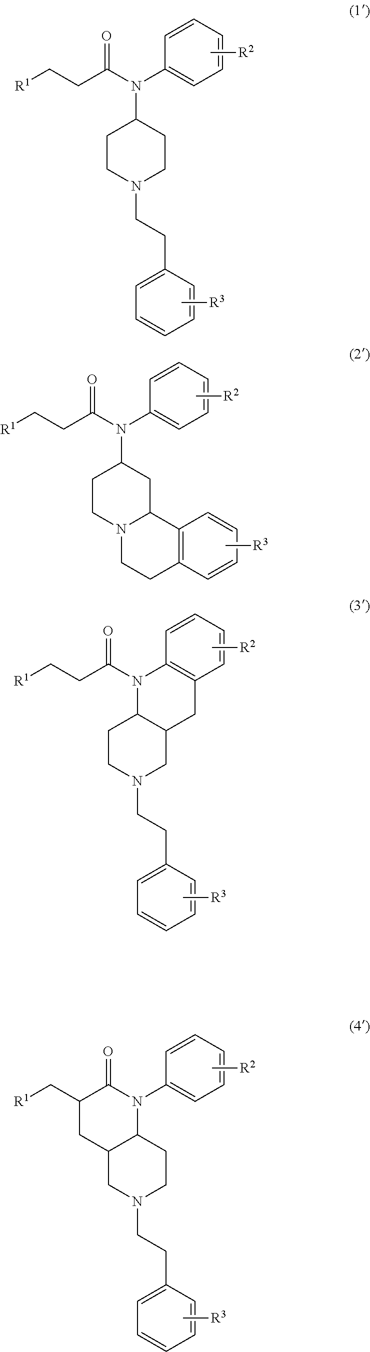

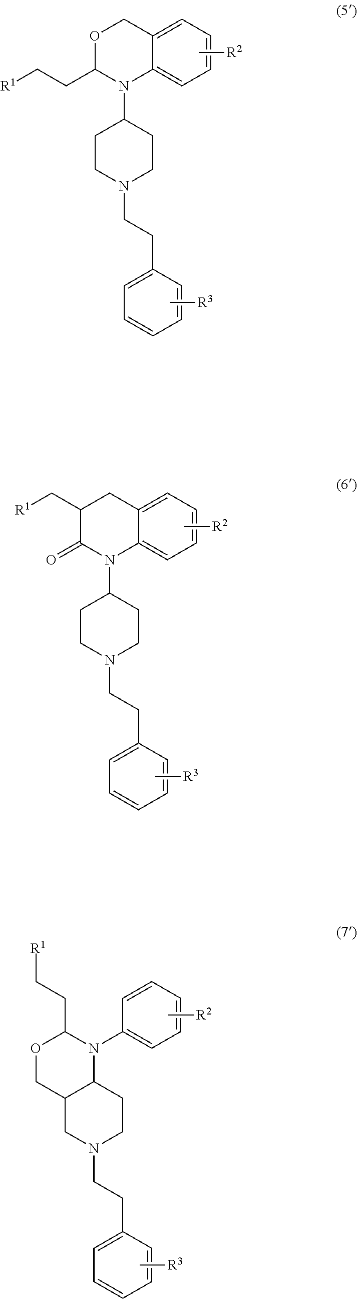

wherein R¹, R², and R³ are each independently H or —(C₁-C₁₂ alkyl)-COOH, provided that at least one of R¹, R², and R³ is —(C₁-C₁₂ alkyl)-COOH. In some aspects of Formulae (1′)-(7′), only one of R¹, R², and R³ is —(C₁-C₁₂ alkyl)-COOH. In some aspects of Formulae (1′)-(7′), R¹ and R² are H, and R³ is —(C₁-C₁₂ alkyl)-COOH. In some aspects of Formulae (1′)-(7′), R¹ and R³ are H, and R² is —(C₁-C₁₂ alkyl)-COOH. In some aspects of Formulae (1′)-(7′), R² and R³ are H, and R¹ is —(C₁-C₁₂ alkyl)-COOH. In any of the foregoing aspects, the alkyl portion of —(C₁-C₁₂ alkyl)-COOH can be a C₂-C₁₂ alkyl or C₃-C₁₂ alkyl, such as a C₂-C₈ alkyl or C₃-C₈ alkyl (e.g., a C₂, C₃, C₄, C₅, C₆, C₇ or C₈ alkyl). Preferably, the alkyl portion of —(C₁-C₁₂ alkyl)-COOH is C₅.

In some aspects, the fentanyl analogue of Formula (1) or (1′) has the structure of one of Formulae (8)-(10):

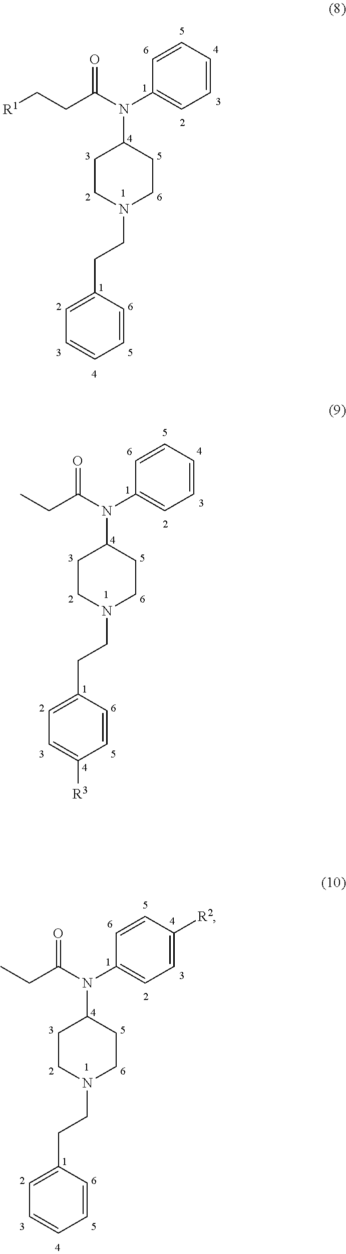

wherein R¹, R², and R³ are each —(C₁-C₁₂ alkyl)-COOH. In some aspects, the alkyl portion of —(C₁-C₁₂ alkyl)-COOH for R¹, R², and/or R³ can be a C₂-C₁₂ alkyl or C₃-C₁₂ alkyl, such as a C₂-C₈ alkyl or C₃-C₈ alkyl (e.g., a C₂, C₃, C₄, C₅, C₆, C₇ or C₈ alkyl).

In some aspects, the fentanyl analogue of Formula (9), (10), or (8) is one of Analogues (1)-(3), respectively:

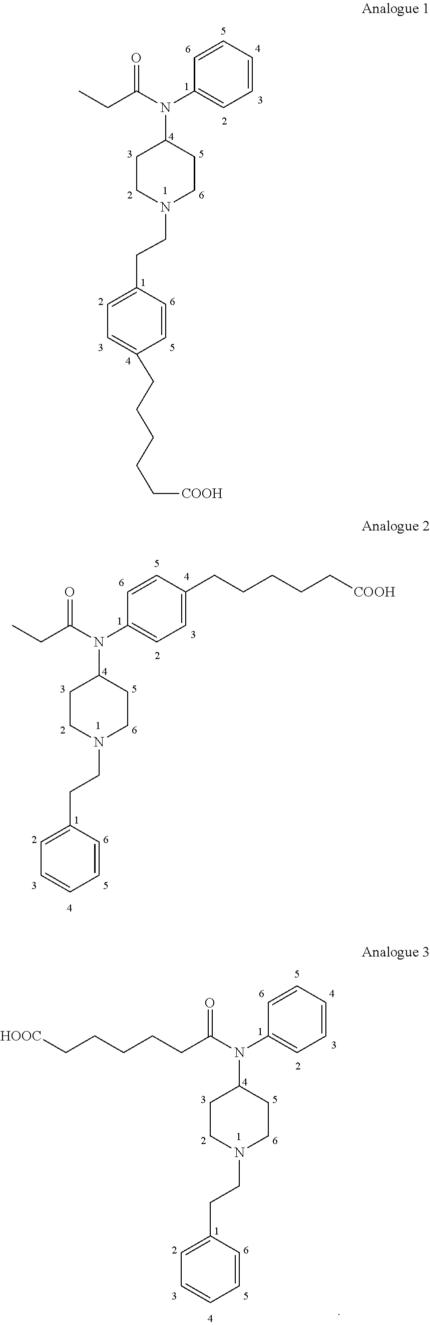 [SMILES: CCC(=O)N(c1ccccc1)C1CCN(CCc2ccc(CCCCCC(=O)O)cc2)CC1 ; CCC(=O)N(c1ccc(CCCCCC(=O)O)cc1)C1CCN(CCc2ccccc2)CC1 ; O=C(O)CCCCCC(=O)N(c1ccccc1)C1CCN(CCc2ccccc2)CC1]

In some aspects, the fentanyl analogue is selected from

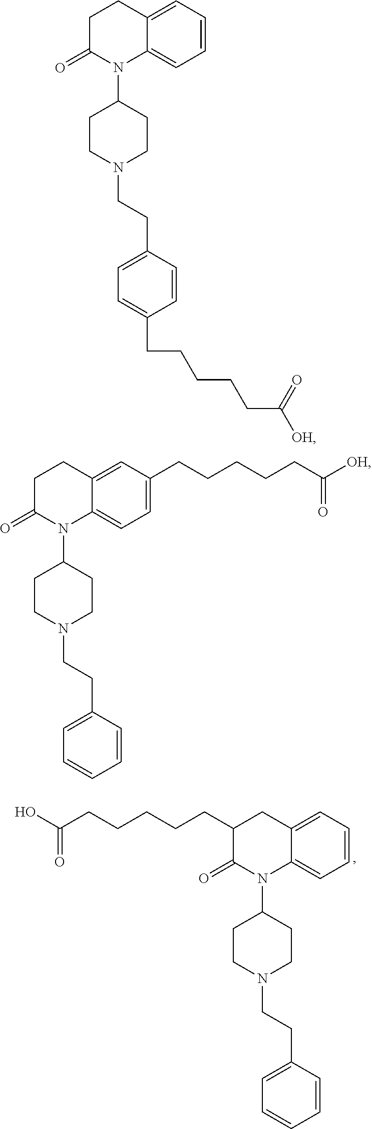

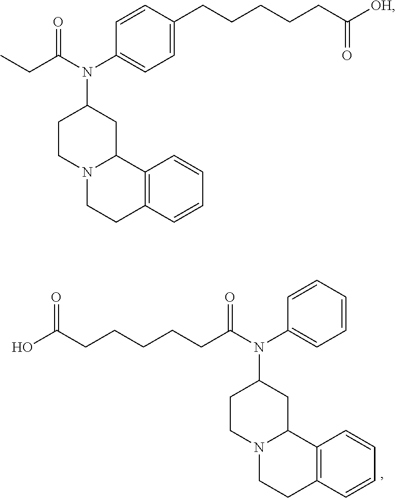 [SMILES: CCC(=O)N(c1ccc(CCCCCC(=O)O)cc1)C1CCN2CCc3ccccc3C2C1 ; O=C(O)CCCCCC(=O)N(c1ccccc1)C1CCN2CCc3ccccc3C2C1]

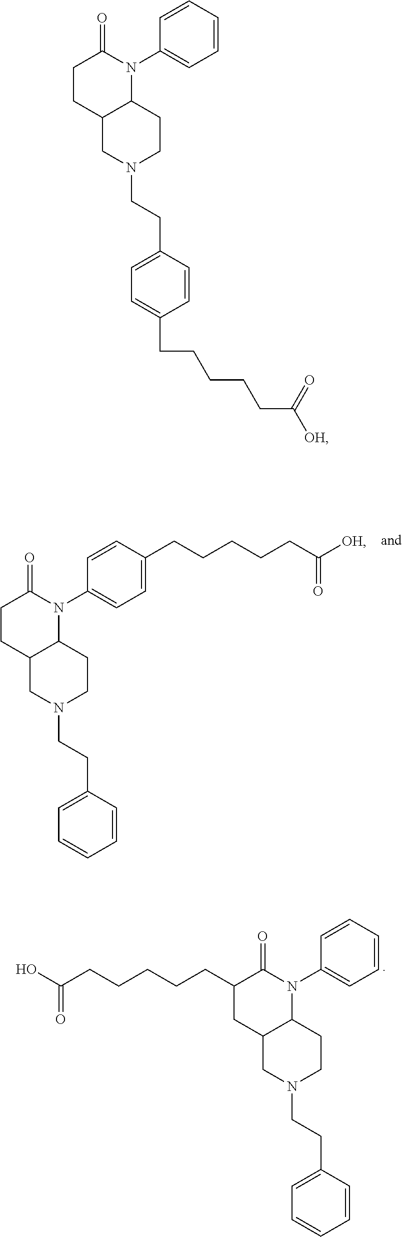 [SMILES: O=C(O)CCCCCc1ccc(CCN2CCC3C(CCC(=O)N3c3ccccc3)C2)cc1 ; O=C(O)CCCCCc1ccc(N2C(=O)CCC3CN(CCc4ccccc4)CCC32)cc1 ; O=C(O)CCCCCC1CC2CN(CCc3ccccc3)CCC2N(c2ccccc2)C1=O]

Without wishing to be bound by any particular theory or mechanism of action, it is believed the particular structure of the fentanyl analogue provides a mechanism of attachment of the molecule to an immunopotent carrier protein in a manner that enables the conjugate to elicit an effective immune response to fentanyl when administered to a subject (e.g., a mammal, such as a human). Thus, the fentanyl analogues are believed to be useful for eliciting an immune response in a subject. Antibodies that bind fentanyl will inhibit or prevent fentanyl from reaching the brain, thus reducing or eliminating addictive reward and inadvertent fentanyl overdose. Thus, the fentanyl analogues and conjugates comprising same are believed to be useful as a vaccine to treat or prevent addiction to fentanyl and/or to reduce the likelihood of overdose, particularly fatal overdose, in a subject.

In general, the fentanyl analogues described herein are synthetically prepared. General methods for preparing analogues of the invention are described herein.

The fentanyl analogue can be part of a conjugate. For instance, the fentanyl analogue can be conjugated to an immune adjuvant (e.g., an immunopotent carrier protein) that enhances the immune response to the fentanyl analogue.

In some aspects, the conjugate comprises, consists essentially of, or consists of a fentanyl analogue and an immunogenic capsid protein of an adenovirus (adenoviral vector, such as a hexon, fiber, or penton base protein, or combination thereof), optionally in combination with other adenoviral proteins or a disrupted adenovirus. The immunogenic capsid protein can be an isolated capsid protein, or the capsid protein can be provided by (or be part of) a non-infectious adenovirus, such as a disrupted adenovirus.

When the conjugate consists essentially of an isolated adenovirus capsid protein conjugated to a fentanyl analogue, additional components can be included that do not materially affect the conjugate (e.g., protein or other moieties, such as biotin, that facilitate purification or isolation). When the conjugate consists of an isolated adenovirus capsid protein coupled to a fentanyl analogue, the conjugate does not comprise any additional components (i.e., components that are not endogenous to the adenovirus capsid protein or endogenous to the disrupted adenovirus when the capsid protein is part of a disrupted adenovirus. By “isolated” is meant the capsid protein is removed from its natural environment. The isolated capsid protein can comprise an intact adenovirus or any portion thereof, so long as the capsid protein is included in the isolate. For example, the isolated capsid protein can be removed from all other adenovirus proteins, with the exception of one or more other coat or capsid proteins (e.g., hexon, fiber protein and/or penton protein). In some aspects, the isolated capsid protein is removed from all adenovirus proteins. By “purified” is meant that the capsid protein, whether it has been removed from nature or synthesized and/or amplified under laboratory conditions, has been increased in purity, wherein “purity” is a relative term, not “absolute purity.” It is to be understood, however, that proteins may be formulated with diluents or adjuvants and still for practical purposes be isolated. For example, proteins can be mixed with an acceptable carrier or diluent when used for introduction into cells or a subject (e.g., a human).

The term “conjugate,” as used herein, refers to a compound comprising two or more molecules (e.g., proteins, carbohydrates, or nucleic acid molecules) that are chemically linked. The two or more molecules desirably are chemically linked using any suitable chemical bond (e.g., covalent bond), and any suitable linking group or moiety. Suitable chemical bonds are well known in the art and include, e.g., disulfide bonds, amide bonds, acid labile bonds, photolabile bonds, peptidase labile bonds, thioether, and esterase labile bonds.

The immunogenic capsid protein can be from any type of adenovirus. Adenovirus (Ad) is a 36 kb double-stranded DNA virus that efficiently transfers DNA in vivo to a variety of different target cell types. While the adenovirus capsid protein can be obtained or derived from a non-human adenovirus (e.g., simian, avian, canine, ovine, or bovine adenoviruses), the capsid protein is preferably a human adenovirus capsid protein. The adenovirus can be of subgroup A (e.g., serotypes 12, 18, and 31), subgroup B (e.g., serotypes 3, 7, 11, 14, 16, 21, 34, 35, and 50), subgroup C (e.g., serotypes 1, 2, 5, and 6), subgroup D (e.g., serotypes 8, 9, 10, 13, 15, 17, 19, 20, 22-30, 32, 33, 36-39, and 42-48), subgroup E (e.g., serotype 4), subgroup F (e.g., serotypes 40 and 41), an unclassified serogroup (e.g., serotypes 49 and 51), or any other adenoviral serotype. Adenoviral serotypes 1 through 51 (i.e., Ad1 through Ad51) are available from the American Type Culture Collection (ATCC, Manassas, VA). In some aspects, the capsid protein is from an adenovirus of human subgroup C, especially serotype 2 or even more desirably serotype 5. In other aspects, the capsid protein is from Ad12 (group A), Ad7 or Ad35 (group B), Ad28 or Ad30 (group D), Ad4 (group E), or Ad41 (group F) adenovirus. Nucleic acid sequences and amino acid sequences of capsid proteins from a variety of adenoviruses are publicly available from the National Center of Biotechnology Information (NCBI) and are disclosed in, for example, Crawford-Miksza et al., J. Virol., 70(3): 1836-1844 (1996). In an aspect, the conjugate comprises a fentanyl analogue, as described herein, and one or more capsid proteins from an Ad5 adenovirus.

In one aspect, the capsid protein is provided by a disrupted adenovirus. A “disrupted” adenovirus is an adenovirus that has been treated with heat and/or one or more detergents so as to render the adenovirus or adenoviral vector non-infectious in mammals. Treating adenoviruses with a mild detergent has been shown to disrupt the viral capsid and to release the nucleoprotein core, groups of nine hexon capsomers, free peripentonal hexons, penton base, and fiber capsomers (see, e.g., Molinier-Frenkel et al., J. Virol., 76: 127-135 (2002), Boulanger et al., J. Gen. Virol., 44: 783-800 (1979), Boulanger, et al., FEBS Lett., 85: 52-56 (1978), and Nermut, The Architecture of Adenoviruses, pp. 5-34, in H. S. Ginsberg (ed.), “The Adenoviruses,” Plenum Press, New York, N.Y. (1984)). The adenovirus can be treated with any suitable detergent known in the art that disrupts the structure of a virus. Examples of such detergents include sodium deoxycholate (DOC), sodium dodecyl sulfate (SDS). An adenovirus can be treated with “heat” by exposing the adenovirus to a temperature above about 50° C., e.g., about 50° C. to about 70° C. The adenovirus can be exposed to a temperature of about 50° C. or higher, about 55° C. or higher, about 60° C. or higher, or about 65° C. or higher. Alternatively, or in addition, the adenovirus can be exposed to a temperature of about 70° C. or lower, about 65° C. or lower, about 60° C. or lower, or about 55° C. or lower. Thus, the adenovirus can be exposed to a temperature between any two of the above endpoints. For example, the adenovirus can be exposed to a temperature of about 50° C. to about 55° C., about 55° C. to 60° C., about 60° C. to about 65° C., about 65° C. to about 70° C. In some aspects, the adenovirus is disrupted by both heat and one or more detergents as described above.

In another aspect, the capsid protein is a capsid protein that is isolated or purified from an adenovirus, or synthetically or recombinantly produced. Capsid proteins can be purified from an intact adenovirus using protein purification methods know in the art (see, e.g., Ausubel et al. (eds.), Short Protocols in Molecular Biology, 5^{th} Ed., John Wiley & Sons, New York (2002)), Such methods include, for example, chromatographic methods (e.g., ion exchange chromatography, ion exchange chromatography, high performance liquid chromatography (HPLC), etc.), immunoprecipitation, and ultracentrifugation. Alternatively, the adenovirus capsid protein can be synthetically or recombinantly produced using routine techniques, such as those described in, for example, Ausubel et al., supra, and Sambrook et al., Molecular Cloning, a Laboratory Manual, 3rd edition, Cold Spring Harbor Press, Cold Spring Harbor, N.Y. (2001).

The adenovirus capsid protein of the inventive conjugate can be a wild-type capsid protein, or can be modified. For instance, the adenovirus capsid protein can be modified in any number of ways to facilitate purification of the adenovirus capsid protein or conjugate comprising same, or to improve the biological activity of the protein or conjugate. By way of further illustration, the adenovirus capsid protein can be labeled and/or tagged in order to facilitate purification. Any suitable protein tag or label can be used in the invention, including, e.g., affinity tags (e.g., a poly (His) tag), solubilization tags (e.g., thioredoxin), chromatography tags (e.g., a FLAG tag), epitope tags (e.g., a c-myc tag), or fluorescence tags. A variety of protein purification tags are known in the art and can be used in the context of the invention (see, e.g., Lichty et al., Protein Expr. Purf., 41(1): 98-105 (2005)).

Exposed lysine residues on an adenovirus capsid protein provide free amine groups that are a target for coupling to carboxylate group-containing antigens, and many of the aforementioned cross-linking reagents react preferentially with lysine residues. Thus, it may be advantageous to add or remove one or more lysine residues to the adenovirus capsid protein as a mechanism to control the level of attachment of fentanyl analogues to the adenovirus capsid protein. In some aspects, the adenovirus capsid protein can comprises one or more non-native lysine residues (e.g., 1 or more, 3 or more, 5 or more, or 7 or more lysine residues). Alternatively, or in addition, the number of non-native lysine residues can be 25 or less, e.g., 20 or less, 15 or less, or 10 or less. Thus, the number of non-native lysine residues can be bounded by any two of the above endpoints. For example, the number of non-native lysine residues can be 1-25, 3-20, 5-10, 5-15, or 7-10. In some aspects, the non-native lysine residues are incorporated into one or more flexible loops of the hexon protein. Coupling can be accomplished via the carboxylic acids of glutamate and aspartate, or via sulfhydryls of cysteine as well. Such modifications can be performed using routine techniques (see, e.g., Sambrook et al., supra; and Ausubel, et al., supra).

The adenovirus capsid protein also can be modified in order to facilitate polymerization or aggregation of the inventive conjugate into larger complexes, which may enhance the immunogenicity of the inventive conjugate in vivo. Methods for modifying proteins to facilitate polymerization or aggregation include, but are not limited to, three-dimensional domain swapping (as described in, e.g., Ogihara et al., Proc. Natl. Acad. Sci. USA, 98(4): 1404-1409 (2001)), oxidation of cysteine residues added to an external protein loop, using bivalent crosslinkers arranged in a “head to toe” manner at the N-terminal and C-terminal of the protein, and treatment of the protein with gluteraldehyde (see, e.g., Migneault et al., BioTechniques, 37: 790-802 (2004)).

The adenovirus capsid protein can be modified in order to enhance the adjuvant effects of the capsid protein. For example, the adenovirus capsid protein can be engineered to contain a peptide adjuvant. A variety of peptide adjuvants that can be recombinantly attached to a protein of interest (e.g., as a fusion protein) are known in the art and include, for example, heat shock protein peptides, peptides of toll-like receptor ligands (TLRs), fibronectin-binding peptide (FBP), and peptides derived from high mobility group box (HMGB1) protein 1. Peptide adjuvants also are described in, e.g., U.S. Patent Application Publication 2011/0305720. One of ordinary skill in the art will appreciate that any combination of the above-described protein modifications can be used in the context of preparing the inventive conjugate.

In still other aspects, the immunogenic capsid protein can be provided by an immunogenic portion of a full length capsid protein. The portion of an adenovirus capsid protein can be of any size, so long as the inventive conjugate can elicit an immune response against fentanyl in a human. A “portion” of an amino acid sequence comprises at least three amino acids (e.g., about 3 to about 1,000 amino acids). Preferably, a “portion” of an amino acid sequence comprises 3 or more (e.g., 5 or more, 10 or more, 15 or more, 20 or more, 25 or more, 30 or more, 40 or more, or 50 or more) amino acids, but 1,000 or less (e.g., 800 or less, 700 or less, 600 or less, 500 or less, 400 or less, 300 or less, 200 or less, or 100 or less) amino acids. Preferably, a portion of an amino acid sequence is about 3 to about 500 amino acids (e.g., about 10, 100, 200, 300, 400, or 500 amino acids), about 3 to about 300 amino acids (e.g., about 20, 50, 75, 95, 150, 175, or 200 amino acids), or about 3 to about 100 amino acids (e.g., about 15, 25, 35, 40, 45, 60, 65, 70, 80, 85, 90, 95, or 99 amino acids), or a range defined by any two of the foregoing values. More preferably, a “portion” of an amino acid sequence comprises no more than about 500 amino acids (e.g., about 3 to about 400 amino acids, about 10 to about 250 amino acids, or about 50 to about 100 amino acids, or a range defined by any two of the foregoing values).

The fentanyl analogue can be conjugated to the adenovirus capsid protein in any suitable manner. For instance, the fentanyl analogue can be coupled to an adenovirus capsid protein using a homo-bifunctional cross-linker, such as glutaraldehyde, DSG, BM[PEO]4, or BS3, which has functional groups reactive towards amine groups or carboxyl groups of an adenovirus capsid protein. In some aspects, the fentanyl analogue is coupled to an adenovirus capsid protein by way of chemical cross-linking using a hetero-bifunctional cross-linker. Hetero-bifunctional cross-linkers include, for example, SMPH (succinimidyl 6-[(beta-maleimidopropionamido)hexanoate]), sulfo-MBS (m-maleimidobenzoyl-N-hydroxysulfosuccinimide ester), sulfo-EMCS (N-epsilon-maleimidocaproyl-oxysulfosuccinimide ester), sulfo-GMBS (N-gamma-maleimidobutyryl-oxysulfosuccinimide ester), sulfo-SIAB (sulfosuccinimidyl (4-iodoacetyl)aminobenzoate), sulfo-SMPB (sulfosuccinimidyl 4-[p-maleimidophenyl]butyrate), sulfo-SMCC (sulfosuccinimidyl 4-(N-maleimidomethyl)cyclohexane-1-carboxylate), SVSB (succinimidyl-(4-vinylsulfone)benzoate), and SIA (N-succinimidyl iodoacetate), which are commercially available from, for example, Pierce Thermo Fisher Scientific (Rockford, IL, USA).

In some aspects, the fentanyl analogue is conjugated to the adenovirus capsid protein using a crosslinker with a succinyl functional moiety (Leopold et al., Hum. Gene Ther., 9: 367-378 (1998) and Miyazawa et al., J. Virol., 73: 6056-6065 (1999)). Examples of linkers comprising a succinyl functional moiety are N-hydroxysulfosuccinimide (sulfo-NHS) and its uncharged analogue N-hydroxysuccinimide (NHS), which are used to convert carboxyl groups to amine-reactive sulfo-NHS esters. The presence of sulfo-NHS esters increases the efficiency of coupling reactions mediated by carbodiimide compounds, such as EDAC (1-ethyl-3-[3-dimethylaminopropyl]carbodiimide hydrochloride; also known as EDC), which couple carboxyl groups to primary amines, and which also can be used in conjunction with sulfo-NHS. Maleimides, which conjugate to sulfhydryl groups, can also be used to conjugate the fentanyl analogue thereof to a capisd protein of an adenovirus.

Thus, in some aspects, the conjugate can comprise the fentanyl analogue coupled to the capsid protein by way of an amide linkage, optionally with an additional linker. In some embodiments, the conjugate can have the structure of any of Formulae (1a)-(7a):

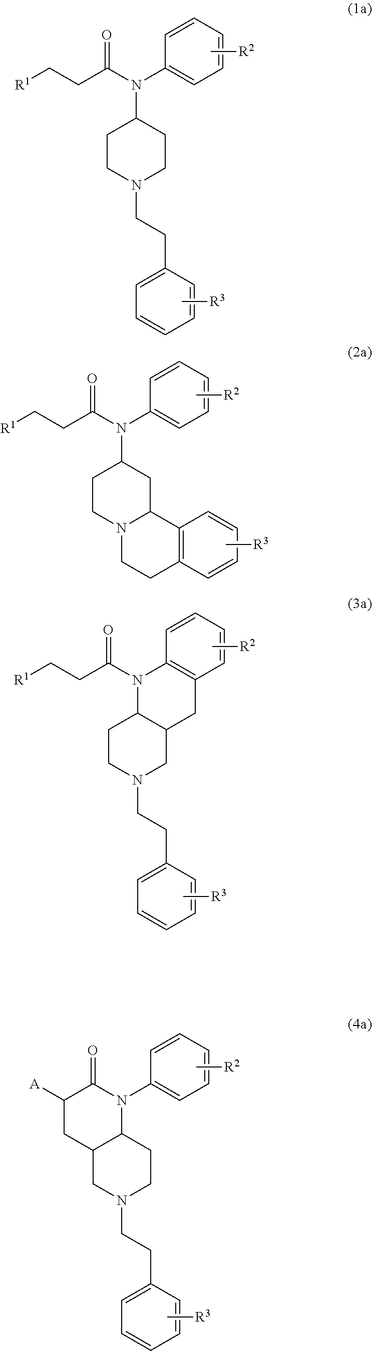

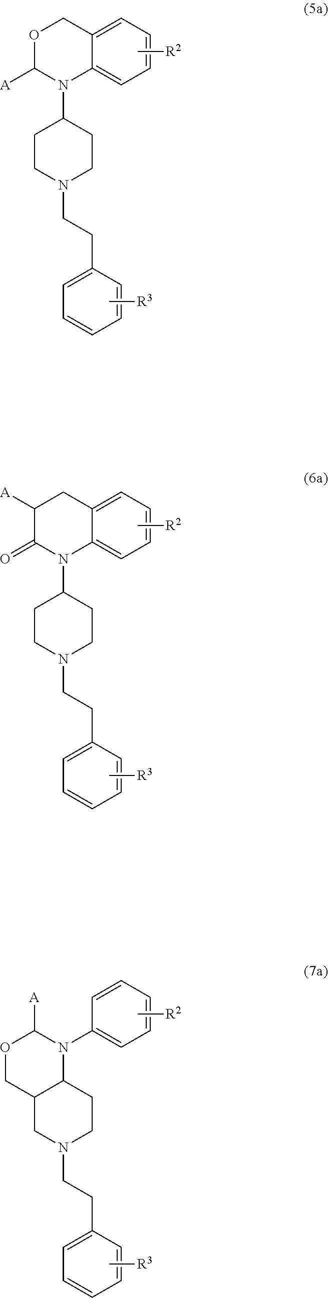

wherein A is H or -alkyl-R¹ and R¹, R², and R³ are each independently H, —(C₁-C₁₂ alkyl)-COOH, or —(C₁-C₁₂ alkyl)-C(O)NH—X, provided that at least one of R¹, R², and R³ is —(C₁-C₁₂ alkyl)-C(O)NH—X, and X is an adenoviral capsid protein. Alternatively or in addition, in some aspects of Formulae (1a)-(7a), A is H. In some aspects of Formulae (1a)-(7a), A is -alkyl-R¹ (e.g., C₁₋₄ alkyl, C₁₋₂ alkyl, C₁-alkyl, or C₂-alkyl), R¹ and R² are H, and R³ is —(C₁-C₁₂ alkyl)-C(O)NH—X. In some aspects of Formulae (1a)-(7a), A is -alkyl-R¹ (e.g., C₁₋₄ alkyl, C₁₋₂ alkyl, C₁-alkyl, or C₂-alkyl), R¹ and R³ are H, and R² is —(C₁-C₁₂ alkyl)-C(O)NH—X. In some aspects of Formulae (1a)-(7a), A is -alkyl-R¹ (e.g., C₁₋₄ alkyl, C₁₋₂ alkyl, C₁-alkyl, or C₂-alkyl), R² and R³ are H, and R¹ is —(C₁-C₁₂ alkyl)-C(O)NH—X. In any of the foregoing aspects, the alkyl portion of —(C₁-C₁₂ alkyl)-COOH and —(C₁-C₁₂ alkyl)-C(O)NH—X can be a C₂-C₁₂ alkyl or C₃-C₁₂ alkyl, such as a C₂-C₈ alkyl or C₃-C₈ alkyl (e.g., a C₂, C₃, C₄, C₅, C₆, C₇ or C₈ alkyl)

Thus, in some aspects, the conjugate can have the structure of any of Formulae (1a′)-(7a′):

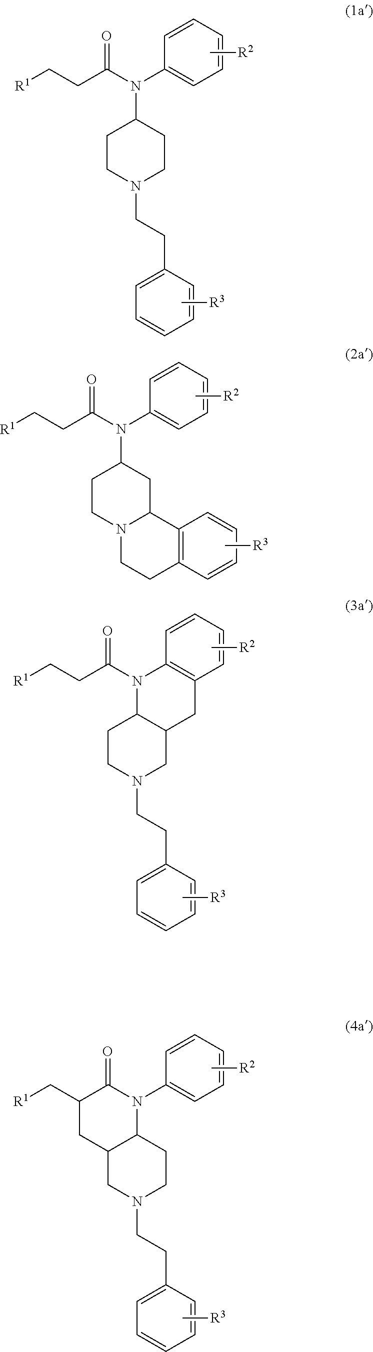

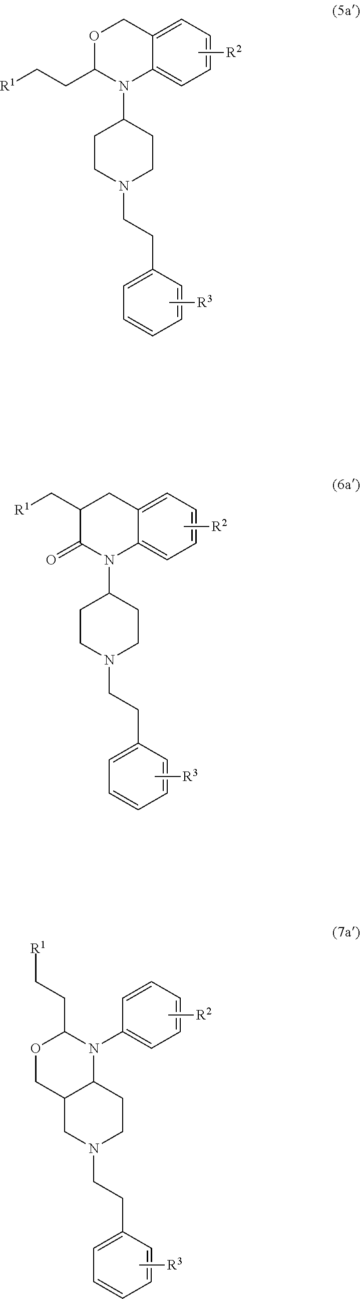

wherein R¹, R², and R³ are each independently H, —(C₁-C₁₂ alkyl)-COOH, or —(C₁-C₁₂ alkyl)-C(O)NH—X, provided that at least one of R¹, R², and R³ is —(C₁-C₁₂ alkyl)-C(O)NH—X, and X is an adenoviral capsid protein. In some aspects of Formulae (1a′)-(7a′), R¹ and R² are H, and R³ is —(C₁-C₁₂ alkyl)-C(O)NH—X. In some aspects of Formulae (1a′)-(7a′), R¹ and R³ are H, and R² is —(C₁-C₁₂ alkyl)-C(O)NH—X. In some aspects of Formulae (1a′)-(7a′), R² and R³ are H, and R¹ is —(C₁-C₁₂ alkyl)-C(O)NH—X. In any of the foregoing aspects, the alkyl portion of —(C₁-C₁₂ alkyl)-COOH and —(C₁-C₁₂ alkyl)-C(O)NH—X can be a C₂-C₁₂ alkyl or C₃-C₁₂ alkyl, such as a C₂-C₈ alkyl or C₃-C₈ alkyl (e.g., a C₂, C₃, C₄, C₅, C₆, C₇ or C₈ alkyl).

In some aspects, the conjugate of Formula (1a) or (1a′) has a structure of any one of Formulae (8a)-(10a):

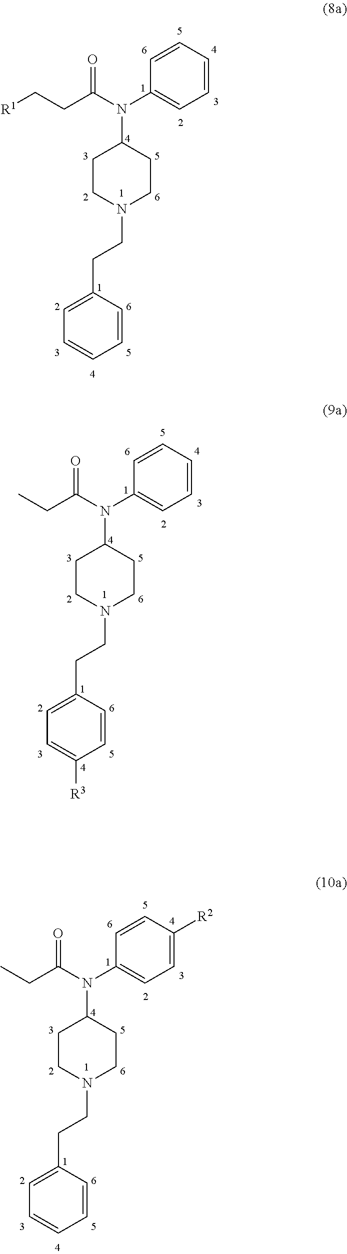

wherein R¹, R², and R³ are each —(C₁-C₁₂ alkyl)-C(O)NH—X, and X is an adenoviral capsid protein. For instance, the conjugate of Formulae (8a)-(10a) can be one of the following:

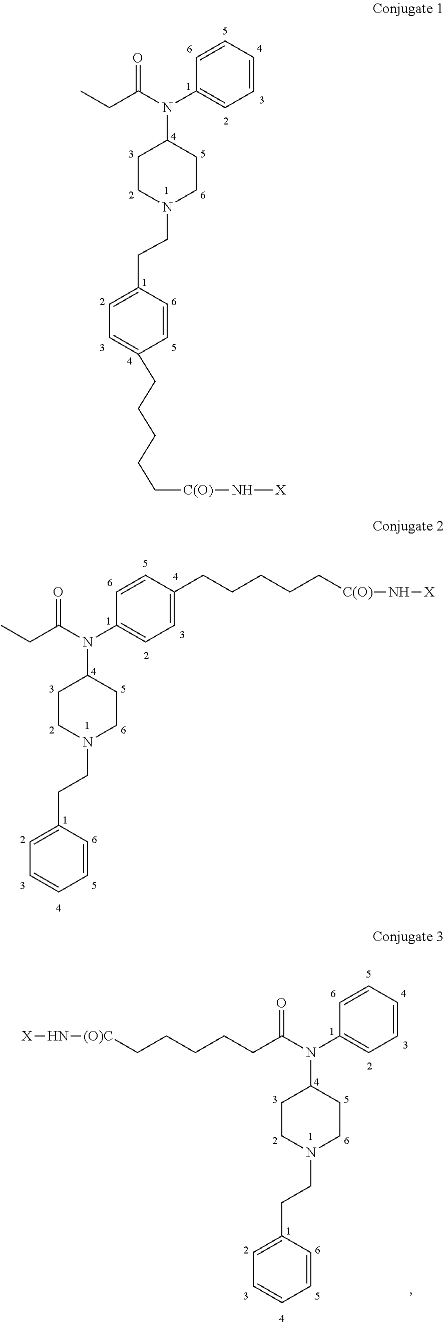

wherein X is an adenoviral capsid protein.

In some aspects, the conjugate can be one of the following:

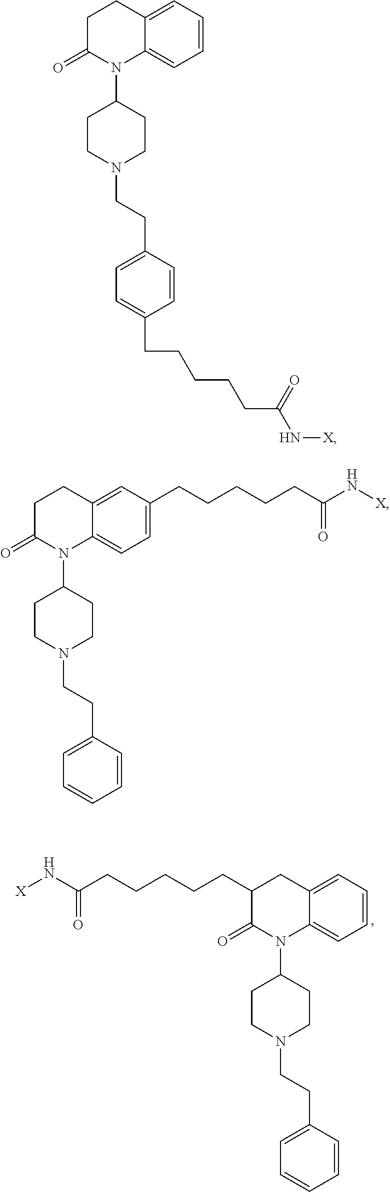

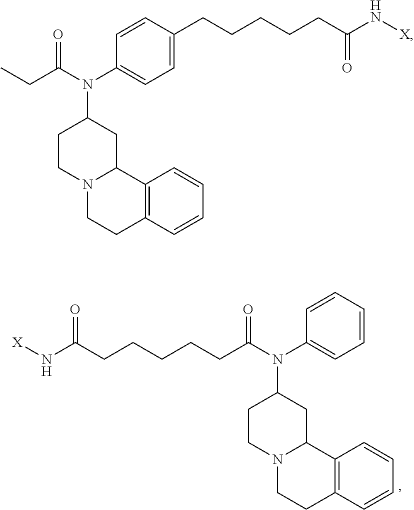

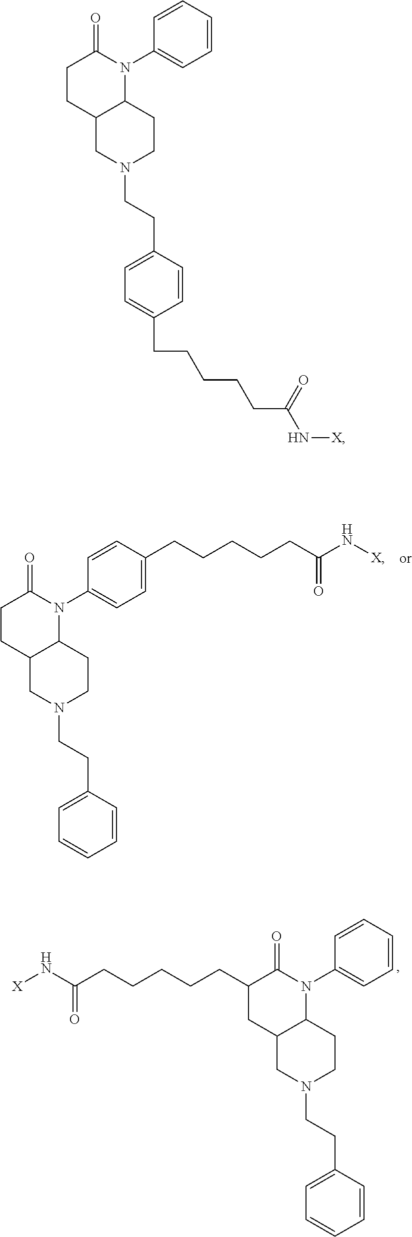

wherein X is an adenoviral capsid protein.

In another aspect, the conjugate comprises carfentanil conjugated to the capsid protein of an adenoviral vector. In some embodiments, the conjugate has the formula:

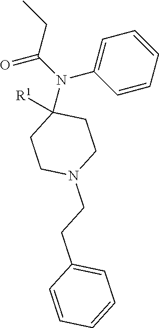

wherein R¹ is —(C₁-C₁₂ alkyl)-C(O)NH—X, and X is an adenoviral capsid protein. The alkyl portion of —(C₁-C₁₂ alkyl)-C(O)NH—X can be a C₂-C₁₂ alkyl or C₃-C₁₂ alkyl, such as a C₂-C₈ alkyl or C₃-C₈ alkyl (e.g., a C₂, C₃, C₄, C₅, C₆, C₇ or C₈ alkyl).

In any of the foregoing structures, X is the capsid protein of an adenoviral vector (e.g., Ad5), such as a hexon, penton base, or fiber protein, or immunogenic fragment thereof, optionally as part of a disrupted adendovirus. When the conjugate is prepared by combining the fentanyl analogue with a disrupted adenovirus (e.g., Ad5), optionally using a homo- or hetero-bifunctionaly cross-linker (e.g., sulpho-NHS and/or EDC), the fentanyl analogue can conjugate to one or more different types of capsid proteins. Furthermore, multiple fentanyl analogue molecules can conjugate to a single protein. Thus, as a related aspect, the disclosure provides a composition comprising a plurality of adenoviral capsid proteins (e.g., a combination of hexon proteins, penton base proteins, and/or fiber protein proteins) conjugated to a fentanyl analogue.

Once the adenovirus capsid protein has been conjugated to the fentanyl analogue, the relative extent of conjugation (also referred to as “conjugation rate”) can be determined by any suitable method, such as Western blotting, mass spectrometry (e.g., matrix-assisted laser desorption/ionization-time of flight mass spectrometry (MALDI-TOF MS)), or by measuring free functional groups on the adenovirus capsid protein by colorimetric assay.

Also provided is a composition comprising, consisting essentially of, or consisting of the above-described a fentanyl analogue described herein and/or a conjugate comprising an isolated adenovirus capsid protein conjugated to a fentanyl analogue and a pharmaceutically acceptable (e.g., physiologically acceptable) carrier. When the composition consists essentially of the inventive conjugate and a pharmaceutically acceptable carrier, additional components can be included that do not materially affect the composition (e.g., adjuvants, buffers, stabilizers, anti-inflammatory agents, solubilizers, preservatives, etc.). When the composition consists of the inventive the fentanyl analogue and/or conjugate and the pharmaceutically acceptable carrier, the composition does not comprise any additional components. Any suitable carrier can be used within the context of the invention, and such carriers are well known in the art. The choice of carrier will be determined, in part, by the particular site to which the composition may be administered and the particular method used to administer the composition. The composition optionally can be sterile. The composition can be frozen or lyophilized for storage and reconstituted in a suitable sterile carrier prior to use. The composition can be generated in accordance with conventional techniques described in, e.g., Remington: The Science and Practice of Pharmacy, 21st Edition, Lippincott Williams & Wilkins, Philadelphia, PA (2001).

Suitable formulations for the composition include aqueous and non-aqueous solutions, isotonic sterile solutions, which can contain anti-oxidants, buffers, and bacteriostats, and aqueous and non-aqueous sterile suspensions that can include suspending agents, solubilizers, thickening agents, stabilizers, and preservatives. The formulations can be presented in unit-dose or multi-dose sealed containers, such as ampules and vials, and can be stored in a freeze-dried (lyophilized) condition requiring only the addition of the sterile liquid carrier, for example, water, immediately prior to use. Extemporaneous solutions and suspensions can be prepared from sterile powders, granules, and tablets of the kind previously described. Preferably, the carrier is a buffered saline solution. More preferably, the inventive conjugate is administered in a composition formulated to protect the conjugate from damage prior to administration. For example, the composition can be formulated to reduce loss of the conjugate on devices used to prepare, store, or administer the conjugate, such as glassware, syringes, or needles. The composition can be formulated to decrease the light sensitivity and/or temperature sensitivity of the conjugate. To this end, the composition preferably comprises a pharmaceutically acceptable liquid carrier, such as, for example, those described above, and a stabilizing agent selected from the group consisting of polysorbate 80, L-arginine, polyvinylpyrrolidone, trehalose, and combinations thereof. Use of such a composition will extend the shelf life of the conjugate, facilitate administration, and increase the efficiency of the inventive method.

The composition also can be formulated to enhance delivery and/or antigen presentation. In addition, one of ordinary skill in the art will appreciate that the inventive the fentanyl analogue and/or conjugate can be present in a composition with other therapeutic or biologically-active agents. For example, factors that control inflammation, such as ibuprofen or steroids, can be part of the composition to reduce swelling and inflammation associated with in vivo administration of the conjugate. Immune system stimulators or adjuvants, e.g., interleukins, lipopolysaccharide, and double-stranded RNA (ribonucleic acid), can be administered to enhance or modify the anti-fentanyl immune response. Antibiotics, i.e., microbicides and fungicides, can be present to treat existing infection and/or reduce the risk of future infection, such as infection associated with drug administration. In some aspects, the composition comprises a carbomer-lecithin adjuvant, such as ADJUPLEX® (Advanced BioAdjuvants, Omaha, NE).

Further provided herein is a method of inducing an immune response against fentanyl in a subject, particularly a mammal (e.g., human), which comprises administering an effective amount of the conjugate or composition comprising same to the subject, whereby the fentanyl analogue is presented to the immune system of the subject to induce an immune response against fentanyl in the subject. In aspects of the invention, the subject is addicted to fentanyl; in other aspects, the subject is not yet addicted to fentanyl, but is being administered fentanyl or is self-administering fentanyl. The conjugate is administered to a subject, whereupon an immune response against fentanyl is induced. The immune response can be a humoral immune response. Ideally, the immune response provides a clinical benefit upon exposure to the antigen. A “clinical benefit” can be, for example, a reduction in the physiological effects of fentanyl, a reduction in the reward or pleasure associated with use of fentanyl, a reduction in the likelihood of regaining or furthering an addiction to fentanyl, reducing the likelihood or preventing (inhibiting or halting or delaying) a new fentanyl addiction, and/or otherwise improving the safety of fentanyl use (e.g., by reducing the likelihood of an overdose or fatal overdose, including overdoses resulting from inadvertent exposure or use of other drugs laced with fentanyl). However, non-clinical uses also are applicable. For instance, the fentanyl analogues and conjugates also can be used for antibody production and harvesting, and the resulting antibodies used for clinical or diagnostic purposes (e.g., to detect the presence of fentanyl in blood).

Any route of administration can be used to deliver the composition to the subject. Indeed, although more than one route can be used to administer the composition, a particular route can provide a more immediate and more effective reaction than another route. Preferably, the composition is administered via injection, especially intramuscular injection. A dose of composition also can be applied or instilled into body cavities, absorbed through the skin (e.g., via a transdermal patch), inhaled, ingested orally, topically applied to tissue, or administered parenterally via, for instance, intravenous, peritoneal, or intraarterial administration.

Formulations suitable for parenteral administration (e.g., injection, infusion) include aqueous and non-aqueous, isotonic sterile injection solutions, which can contain anti-oxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. The fentanyl analogues and conjugates can be administered in a physiologically acceptable diluent in a pharmaceutical carrier, such as a sterile liquid or mixture of liquids, including water, saline, aqueous dextrose and related sugar solutions, an alcohol, such as ethanol, isopropanol, or hexadecyl alcohol, glycols, such as propylene glycol or polyethylene glycol, glycerol ketals, such as 2,2-dimethyl-1,3-dioxolane-4-methanol, ethers, such as poly(ethyleneglycol) 400, an oil, a fatty acid, a fatty acid ester or glyceride, or an acetylated fatty acid glyceride with or without the addition of a pharmaceutically acceptable surfactant, such as a soap or a detergent, suspending agent, such as pectin, carbomers, methylcellulose, hydroxypropylmethylcellulose, or carboxymethylcellulose, or emulsifying agents and other pharmaceutical adjuvants.

Oils, which can be used in parenteral formulations include petroleum, animal, vegetable, or synthetic oils. Specific examples of oils include peanut, soybean, sesame, cottonseed, corn, olive, petrolatum, and mineral. Suitable fatty acids for use in parenteral formulations include oleic acid, stearic acid, and isostearic acid. Ethyl oleate and isopropyl myristate are examples of suitable fatty acid esters. Suitable soaps for use in parenteral formulations include fatty alkali metal, ammonium, and triethanolamine salts, and suitable detergents include (a) cationic detergents such as, for example, dimethyl dialkyl ammonium halides, and alkyl pyridinium halides, (b) anionic detergents such as, for example, alkyl, aryl, and olefin sulfonates, alkyl, olefin, ether, and monoglyceride sulfates, and sulfosuccinates, (c) nonionic detergents such as, for example, fatty amine oxides, fatty acid alkanolamides, and polyoxyethylene-polypropylene copolymers, (d) amphoteric detergents such as, for example, alkyl-beta-aminopropionates, and 2-alkyl-imidazoline quaternary ammonium salts, and (3) mixtures thereof.

The parenteral formulations will typically contain from about 0.5 to about 25% by weight of the inhibitors in solution. Suitable preservatives and buffers can be used in such formulations. In order to minimize or eliminate irritation at the site of injection, such compositions can contain one or more nonionic surfactants having a hydrophile-lipophile balance (HLB) of from about 12 to about 17. The quantity of surfactant in such formulations ranges from about 5 to about 15% by weight. Suitable surfactants include polyethylene sorbitan fatty acid esters, such as sorbitan monooleate and the high molecular weight adducts of ethylene oxide with a hydrophobic base, formed by the condensation of propylene oxide with propylene glycol. The parenteral formulations can be presented in unit-dose or multi-dose sealed containers, such as ampoules and vials, and can be stored in a freeze-dried (lyophilized) condition requiring only the addition of the sterile liquid carrier, for example, water, for injections, immediately prior to use. Extemporaneous injection solutions and suspensions can be prepared from sterile powders, granules, and tablets of the kind previously described.

The requirements for effective pharmaceutical carriers for injectable compositions are well known to those of ordinary skill in the art. See Pharmaceutics andPharmacy Practice, J. B. Lippincott Co., Philadelphia, Pa., Banker and Chalmers, eds., pages 238-250 (1982), and ASHP Handbook on Injectable Drugs, Toissel, 4th ed., pages 622-630 (1986).

The fentanyl analogues and conjugates can be administered orally to a subject in need thereof. Formulations suitable for oral administration can consist of (a) liquid solutions, such as an effective amount of the compound dissolved in diluents, such as water, saline, or orange juice and include an additive, such as cyclodextrin (e.g., α-, β-, or γ-cyclodextrin, hydroxypropyl cyclodextrin) or polyethylene glycol (e.g., PEG400); (b) capsules, sachets, tablets, lozenges, and troches, each containing a predetermined amount of the active ingredient, as solids or granules; (c) powders; (d) suspensions in an appropriate liquid; and (e) suitable emulsions and gels. Liquid formulations may include diluents, such as water and alcohols, for example, ethanol, benzyl alcohol, and the polyethylene alcohols, either with or without the addition of a pharmaceutically acceptable surfactant, suspending agent, or emulsifying agent. Capsule forms can be of the ordinary hard- or soft-shelled gelatin type containing, for example, surfactants, lubricants, and inert fillers, such as lactose, sucrose, calcium phosphate, and cornstarch. Tablet forms can include one or more of lactose, sucrose, mannitol, corn starch, potato starch, alginic acid, microcrystalline cellulose, acacia, gelatin, guar gum, colloidal silicon dioxide, croscarmellose sodium, talc, magnesium stearate, calcium stearate, zinc stearate, stearic acid, and other excipients, colorants, diluents, buffering agents, disintegrating agents, moistening agents, preservatives, flavoring agents, and pharmacologically compatible carriers. Lozenge forms can comprise the active ingredient in a flavor, usually sucrose and acacia or tragacanth, as well as pastilles comprising the active ingredient in an inert base, such as gelatin and glycerin, or sucrose and acacia, emulsions, gels, and the like containing, in addition to the active ingredient, such carriers as are known in the art.

The fentanyl analogues and conjugates, alone or in combination with other suitable components, can be made into aerosol formulations to be administered via inhalation. These aerosol formulations can be placed into pressurized acceptable propellants, such as dichlorodifluoromethane, propane, nitrogen, and the like. They also may be formulated as pharmaceuticals for non-pressured preparations, such as in a nebulizer or an atomizer.

The composition can be administered in or on a device that allows controlled or sustained release, such as a sponge, biocompatible meshwork, mechanical reservoir, or mechanical implant. Implants (see, e.g., U.S. Pat. No. 5,443,505), devices (see, e.g., U.S. Pat. No. 4,863,457), such as an implantable device, e.g., a mechanical reservoir or an implant or a device comprised of a polymeric composition, are particularly useful for administration of the fentanyl analogue or conjugate. The composition also can be administered in the form of sustained-release formulations (see, e.g., U.S. Pat. No. 5,378,475) comprising, for example, gel foam, hyaluronic acid, gelatin, chondroitin sulfate, a polyphosphoester, such as bis-2-hydroxyethyl-terephthalate (BHET), and/or a polylactic-glycolic acid.

The dose of the conjugate in the composition administered to the subject will depend on a number of factors, including the size (mass) of the subject, the extent of any side-effects, the particular route of administration, and the like. Preferably, the inventive method comprises administering a “therapeutically effective amount” of the composition comprising the inventive conjugate described herein. A “therapeutically effective amount” refers to an amount effective, at dosages and for periods of time necessary, to achieve a desired therapeutic result in a subject, cell, or tissue to be treated.

The therapeutically effective amount may vary according to factors such as the degree of fentanyl addiction, age, sex, and weight of the individual, and the ability of the conjugate to elicit a desired response in the individual. In another aspect, the inventive method can comprise administering a “prophylactically effective amount” of the composition comprising the inventive conjugate. A “prophylactically effective amount” refers to an amount effective, at dosages and for periods of time necessary, to achieve a desired prophylactic result (e.g., prevention of fentanyl addiction). Various general considerations taken into account in determining the “effective amount” are known to those of skill in the art and are described, e.g., in Gilman et al., eds., Goodman and Gilman's. The PharmacologicalBases of Therapeutics, 8th ed., Pergamon Press, 1990; and Remington: The Science and Practice ofPharmacy, 21stEdition, Lippincott Williams & Wilkins, Philadelphia, PA (2001), each of which is herein incorporated by reference.

In an aspect, a typical dose of conjugate in the composition required to achieve a particular therapeutic or prophylactic effect (e.g., prevention or treatment of fentanyl addiction) can be, for example, in the range of 0.001 to 1000 μg; however, doses below or above this exemplary range are within the scope of the invention.

In another aspect, the dose of the conjugate desirably comprises about 0.01 mg per kilogram (kg) of the body weight of the subject (mg/kg) or more (e.g., about 0.05 mg/kg or more, 0.1 mg/kg or more, 0.5 mg/kg or more, 1 mg/kg or more, 2 mg/kg or more, 5 mg/kg or more, 10 mg/kg or more, 15 mg/kg or more, 20 mg/kg or more, 30 mg/kg or more, 40 mg/kg or more, 50 mg/kg or more, 75 mg/kg or more, 100 mg/kg or more, 125 mg/kg or more, 150 mg/kg or more, 175 mg/kg or more, 200 mg/kg or more, 225 mg/kg or more, 250 mg/kg or more, 275 mg/kg or more, 300 mg/kg or more, 325 mg/kg or more, 350 mg/kg or more, 375 mg/kg or more, 400 mg/kg or more, 425 mg/kg or more, 450 mg/kg or more, or 475 mg/kg or more) per day. Typically, the dose will be about 500 mg/kg or less (e.g., about 475 mg/kg or less, about 450 mg/kg or less, about 425 mg/kg or less, about 400 mg/kg or less, about 375 mg/kg or less, about 350 mg/kg or less, about 325 mg/kg or less, about 300 mg/kg or less, about 275 mg/kg or less, about 250 mg/kg or less, about 225 mg/kg or less, about 200 mg/kg or less, about 175 mg/kg or less, about 150 mg/kg or less, about 125 mg/kg or less, about 100 mg/kg or less, about 75 mg/kg or less, about 50 mg/kg or less, about 40 mg/kg or less, about 30 mg/kg or less, about 20 mg/kg or less, about 15 mg/kg or less, about 10 mg/kg or less, about 5 mg/kg or less, about 2 mg/kg or less, about 1 mg/kg or less, about 0.5 mg/kg or less, or about 0.1 mg/kg or less). Any two of the foregoing endpoints can be used to define a close-ended range, or a single endpoint can be used to define an open-ended range.

In an aspect, the composition is administered to the subject two or more (e.g., 2, 3, 4, 5, 6, 7, 8, 9, 10, etc.) times during a therapeutic period. The term “therapeutic period” refers to any suitable time period for the treatment method to be administered to a subject. The therapeutic period can be, for example, 1 day or more and often is 1 week (7 days) or more (e.g., 2 weeks or more, 3 weeks or more, 4 weeks or more, 5 weeks or more, 6 weeks or more, 7 weeks or more, 8 weeks or more, 9 weeks or more, 10 weeks or more, or 11 weeks or more). Alternatively, or in addition, the therapeutic period can be 12 weeks or less (e.g., 11 weeks or less, 10 weeks or less, 9 weeks or less, 8 weeks or less, 7 weeks or less, 6 weeks or less, 5 weeks or less, 4 weeks or less, 3 weeks or less, 2 weeks or less, 1 week or less, 5 days or less, 4 days or less, 3 days or less, or 2 days or less). Any two of the foregoing endpoints can be used to define a close-ended range, or a single endpoint can be used to define an open-ended range.

For purposes of the present invention, the term “subject” preferably is directed to a mammal. Mammals include, but are not limited to, the order Rodentia, such as mice, and the order Lagomorpha, such as rabbits. It is preferred that the mammals are from the order Carnivora, including Felines (cats) and Canines (dogs). It is more preferred that the mammals are from the order Artiodactyla, including Bovines (cows) and Swines (pigs) or of the order Perissodactyla, including Equines (horses). It is most preferred that the mammals are of the order Primates, Cebids, or Simioids (monkeys) or of the order Anthropoids (humans and apes). An especially preferred mammal is a human.

The invention is further illustrated by the following aspects.

Aspect 1. A fentanyl analogue having the structure of any of Formulae (1)-(7).

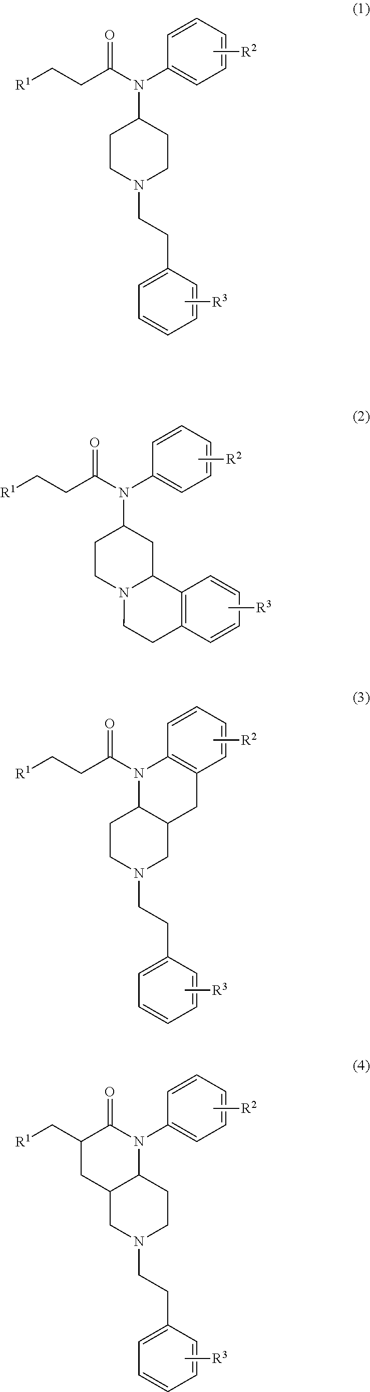

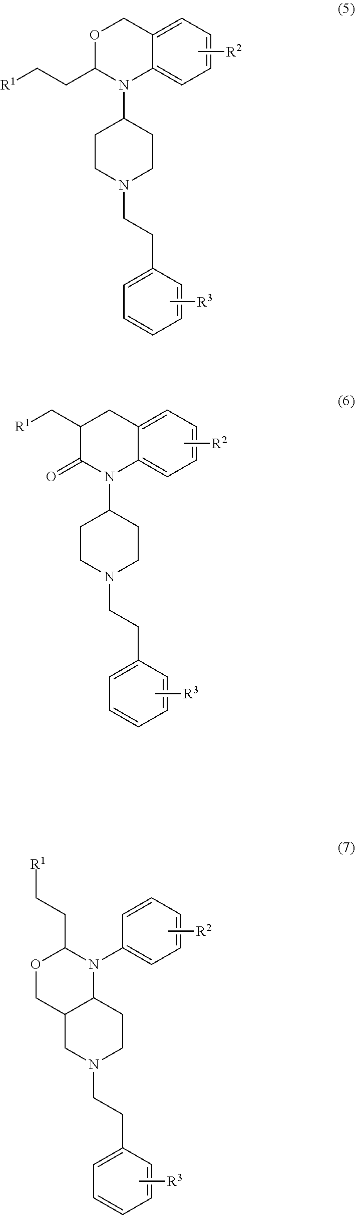

wherein R¹, R², and R³ are each independently H or —(C₁-C₁₂ alkyl)-COOH, provided that at least one of R¹, R², and R³ is —(C₁-C₁₂ alkyl)-COOH;
or having the structure of any one of Formulae (8)-(10):

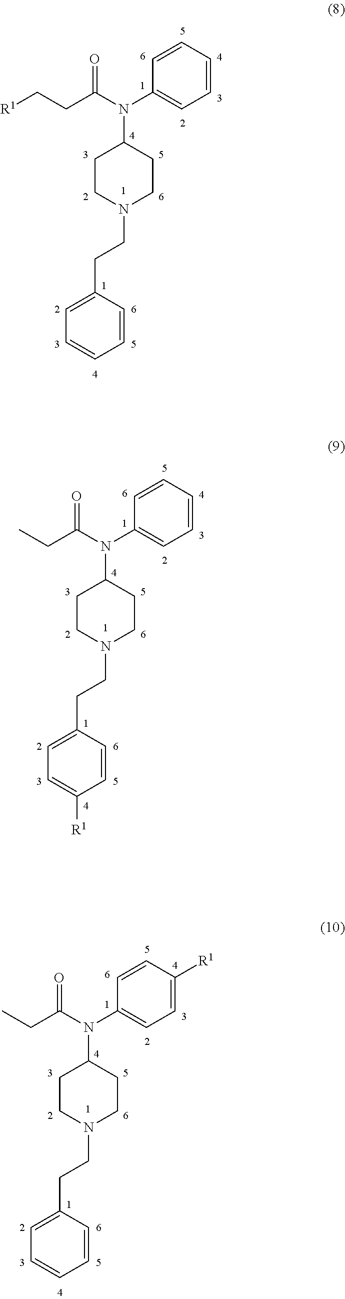

wherein R¹ is —(C₁-C₂ alkyl)-COOH.

Aspect 2. The fentanyl analogue of aspect 1, having the following structure:

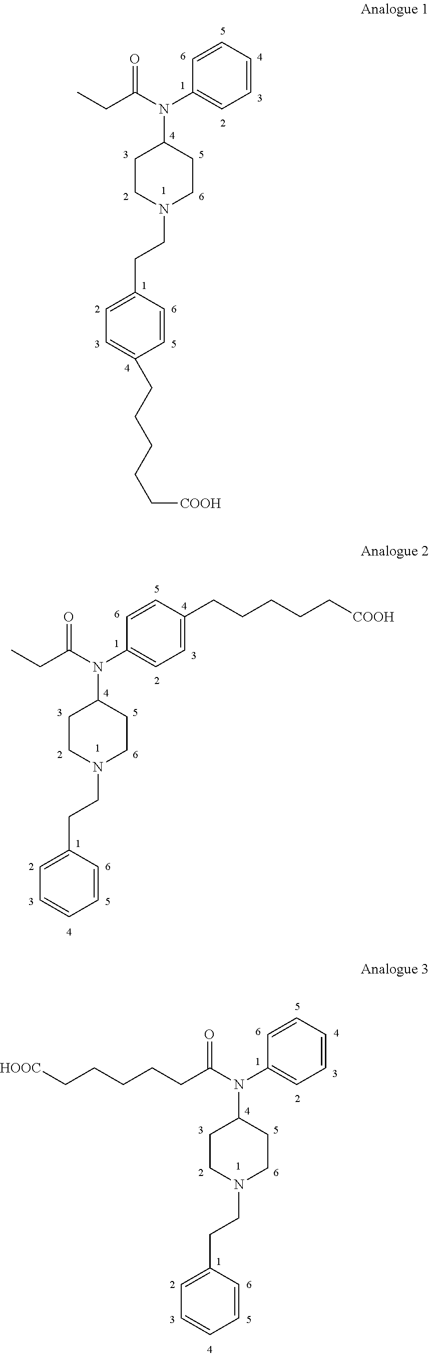 [SMILES: CCC(=O)N(c1ccccc1)C1CCN(CCc2ccc(CCCCCC(=O)O)cc2)CC1 ; CCC(=O)N(c1ccc(CCCCCC(=O)O)cc1)C1CCN(CCc2ccccc2)CC1 ; O=C(O)CCCCCC(=O)N(c1ccccc1)C1CCN(CCc2ccccc2)CC1]

Aspect 3. A conjugate comprising the fentanyl analogue of aspect 1 or 2 conjugated to an adenoviral capsid protein, optionally a hexon protein, penton base protein, or fiber protein.

Aspect 4. The conjugate of aspect 3 having the following structure of any of Formulae (1a)-(7a)-.

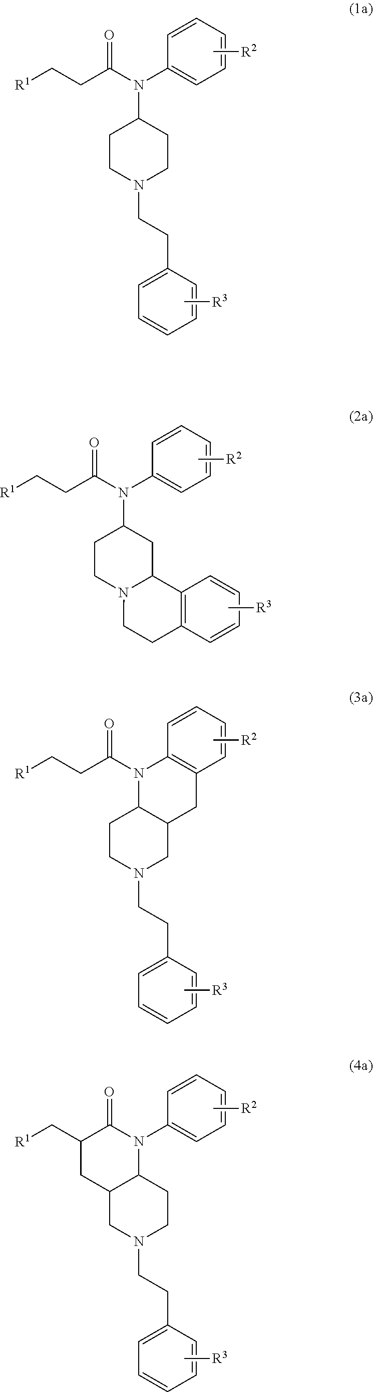

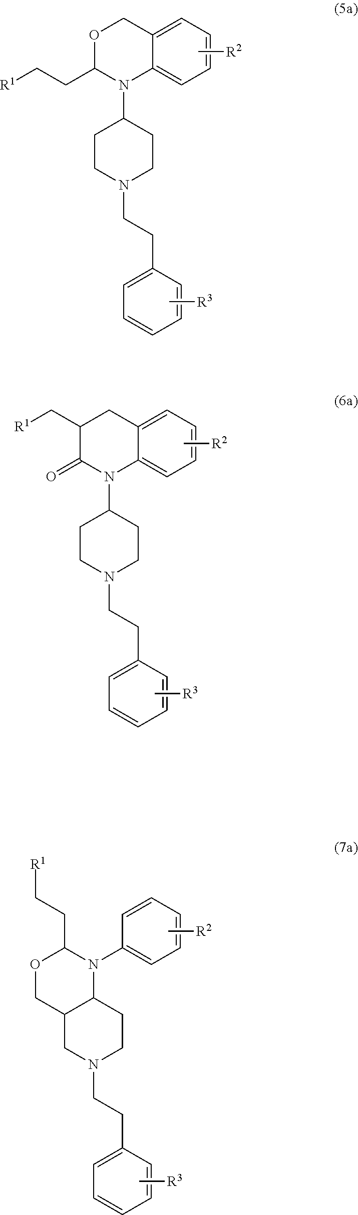

wherein R¹, R², and R³ are each independently H, —(C₁-C₁₂ alkyl)-COOH, or —(C₁-C₁₂ alkyl)-C(O)NH—X, provided that at least one of R¹, R², and R³ is —(C₁-C₁₂ alkyl)-C(O)NH—X, and X is an adenoviral capsid protein;
or having the structure of any one of Formulae (8a)-(10a):

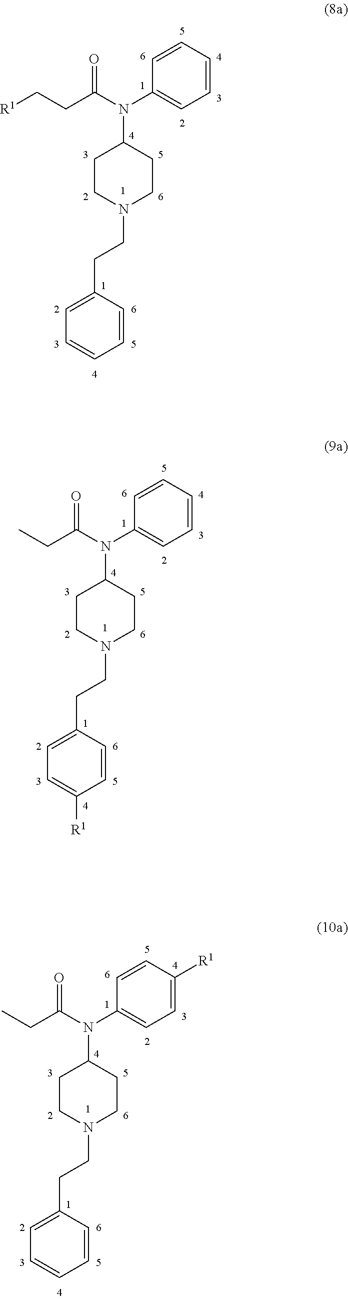

wherein R¹ is —(C₁-C₁₂ alkyl)-C(O)NH—X, and X is an adenoviral capsid protein;
or having the structure:

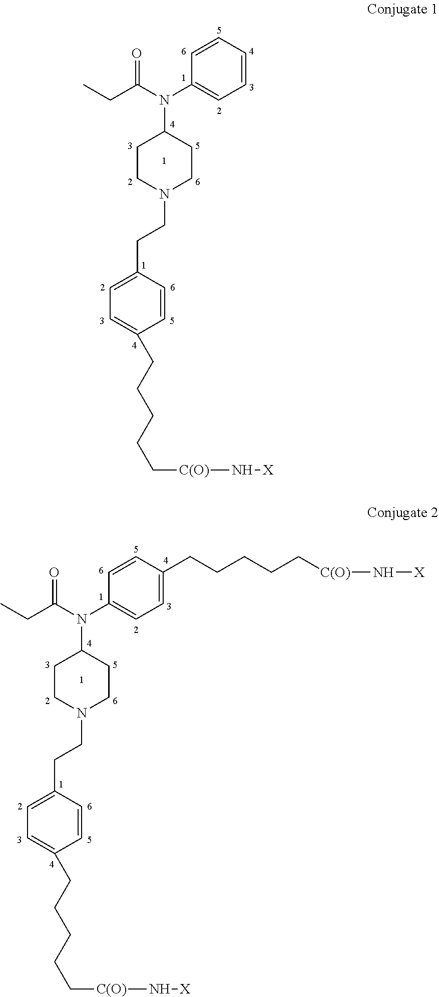

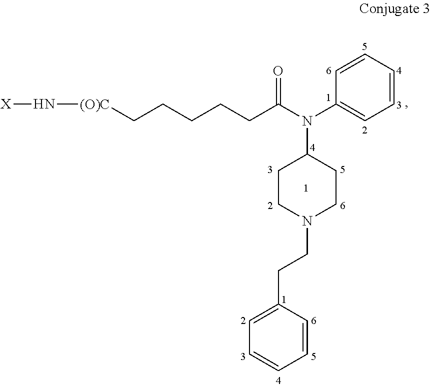

wherein X is an adenoviral capsid protein.

Aspect 5. The conjugate of aspect 3 or 4, wherein the adenovirus capsid protein is isolated or purified.

Aspect 6. The conjugate of aspect 3 or 4, wherein the adenovirus capsid protein is synthetic or recombinant.

Aspect 7. The conjugate of aspect 3 or 4, wherein the conjugate comprises a disrupted adenovirus, or one or more additional adenovirus capsid proteins.

Aspect 8. The conjugate of any one of aspects 3-7, wherein the adenovirus is a human adenovirus, optionally a serotype 5 adenovirus.

Aspect 9. The conjugate of any one of aspects 7 or 8, wherein the conjugate comprises an adenovirus disrupted by exposure to heat and/or detergents.

Aspect 10. A composition comprising the fentanyl analogue or conjugate of any one of aspects 1-9 and a pharmaceutically acceptable carrier.

Aspect 11. A method of inducing an immune response against fentanyl in a subject, which method comprises administering to the subject the conjugate of any of aspects 3-9, or the composition of aspect 10.

Aspect 12. The method of aspect 11, wherein the subject is a mammal or human.

Aspect 13. The method of aspect 11 or 12, wherein the composition is administered to the mammal two or more times during a therapeutic period.

Aspect 14. A method of preparing a conjugate comprising the fentanyl analogue of aspect 1 or 2 and an adenovirus capsid protein, the method comprising combining the fentanyl analogue with an adenovirus capsid protein, whereby the fentanyl analogue is conjugated to the capsid protein.

Aspect 15. The method of aspect 14, wherein the fentanyl analogue and the capsid protein are combined in the presence of N-hydroxysulfosuccinimide (sulfo-NHS) and/or 1-ethyl-3-[3-dimethylaminopropyl]carbodiimide hydrochloride (EDC).

Aspect 16. The method of aspect 14, wherein the method comprises combining the fentanyl analogue with a disrupted adenovirus, and the fentanyl analogue conjugates with a capsid protein of the disrupted adenovirus.

The examples provided herein as attachments further illustrate the invention but, of course, should not be construed as in any way limiting its scope.

### Example 1

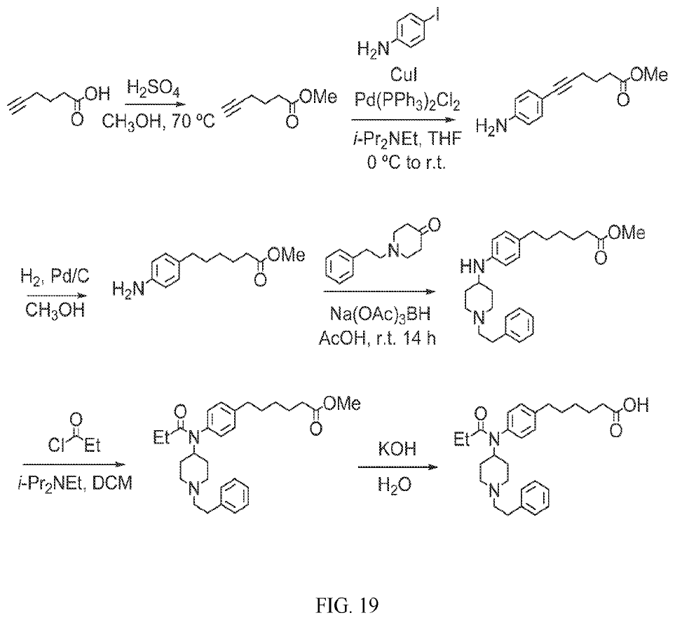

This example demonstrates a chemical synthesis of 6-(4-(N-(1-phenethylpiperidin-4-yl)propionamido)phenyl)hexanoic acid (Analogue 2). FIG. 19 provides a schematic illustration of the chemical synthesis.

### Methyl hex-5-ynoate

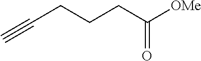

Sulphuric acid (0.6 mL, 11.1 mmol) was added dropwise to methanol (10.8 mL, 26.8 mmol) at 0° C., the resulting solution was stirred for 10 minutes. 5-hexynoic acid (1.01 mL, 8.92 mmol) was added dropwise, and the solution was stirred at reflux (65° C.) overnight. The resulting solution was cooled and concentrated in vacuo, and quenched with water, then extracted with diethyl ether. The organic phase was washed sequentially with an aqueous solution of NaHCO₃, and brine. The organic phase was dried (Na₂SO₄) and concentrated yielding the title compound as a colorless oil (1.02 g, 91% yield); ¹H NMR (500 MHz, CDCl₃) δ 3.68 (s, 3H), 2.46 (t, J=7.4 Hz, 2H), 2.27 (td, J=6.9, 2.4 Hz, 2H), 1.97 (q, J=2.6 Hz, 1H), 1.85 (p, J=7.2 Hz, 2H); ¹³C NMR (126 MHz, CDCl₃) δ 173.66, 83.38, 69.24, 51.73, 32.79, 23.74, 17.99.

### Methyl 6-(4-aminophenyl)hex-5-ynoate

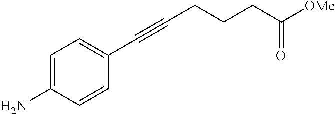 [SMILES: COC(=O)CCCC#Cc1ccc(N)cc1]

To a stirring solution of CuI (42 mg, 0.22 mmol), Pd(PPh₃)₂Cl₂ (78 mg, 0.11 mmol), and 4-iodoaniline (811 mg, 3.70 mmol) in THE (3 mL) at 0° C. was added i-Pr₂NEt (1.3 mL) followed by a solution of methyl hex-5-ynoate (514 mg, 4.07 mmol) in THE (2 mL). The resulting solution was stirred at room temperature overnight. The solution was quenched with an aqueous solution of NH₄Cl, and extracted with EtOAc. The organic layer was washed with brine and dried with Na₂SO₄. The mixture was purified by flash column chromatography and the title compound was obtained as a yellow oil (584 mg, 66% yield); ¹H NMR (500 MHz, CDCl₃) δ 7.19 (d, J=8.5 Hz, 2H), 6.58 (d, J=8.4 Hz, 2H), 3.76 (s, 2H), 3.68 (s, 3H), 2.50 (t, J=7.5 Hz, 2H), 2.45 (t, J=6.9 Hz, 2H), 1.91 (p, J=7.2 Hz, 2H); ¹³C NMR (126 MHz, CDCl₃) δ 173.91, 146.16, 132.92, 114.87, 113.43, 86.38, 81.83, 51.72, 33.10, 24.24, 19.08.

### Methyl 6-(4-aminophenyl)hexanoate

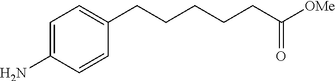 [SMILES: COC(=O)CCCCCc1ccc(N)cc1]

To a solution of methyl 6-(4-aminophenyl)hex-5-ynoate (200 mg, 0.92 mmol) in methanol (5 mL) was added Pd/C (20 mg, 10% w/w). The mixture was then placed under a hydrogen atmosphere and stirred overnight. The mixture was filtered over celite and the filtrate concentrated to obtain the title compound as a dark brown oil (201 mg, 99% yield); ¹H NMR (500 MHz, CDCl₃) δ 6.96 (d, J=8.4 Hz, 2H), 6.62 (d, J=8.4 Hz, 2H), 3.66 (s, 3H), 3.54 (s, 2H), 2.50 (t, J=7.7 Hz, 2H), 2.30 (t, J=7.6 Hz, 2H), 1.64 (p, J=7.6 Hz, 2H), 1.57 (p, J=7.6 Hz, 2H), 1.39-1.30 (m, 2H).

### Methyl 6-(4-((1-phenethylpiperidin-4-yl)amino)phenyl)hexanoate

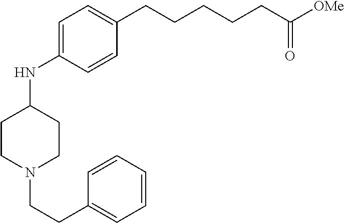 [SMILES: COC(=O)CCCCCc1ccc(NC2CCN(CCc3ccccc3)CC2)cc1]

To a solution of methyl 6-(4-aminophenyl)hexanoate (430.0 mg, 1.94 mmol) in methylene chloride (7.5 mL) at 0° C. was added acetic acid (0.11 mL, 1.94 mmol). To the mixture, N-phenylethylpiperidin-4-one (395.0 mg, 1.94 mmol) was added as a solution in methylene chloride (3.0 mL), followed by the portionwise addition of sodium triacetoxyborohydride (453.0 mg, 2.14 mmol, 1.1 equiv.). The reaction mixture was stirred at ambient temperature for 14 h. After this time, methanol was added and stirred for a further hour. The mixture was quenched with saturated NaHCO₃ aqueous solution and methylene chloride was added. The organic phase was washed with brine, dried over Na₂SO₄, and concentrated in vacuo. The crude mixture was purified by flash column chromatography to give title compound as a light yellow oil (78%); ¹H NMR (500 MHz, Chloroform-d) δ 7.31-7.26 (m, 2H), 7.23-7.15 (m, 3H), 6.96 (d, J=8.3 Hz, 2H), 6.53 (d, J=8.4 Hz, 2H), 3.65 (s, 3H), 3.35 (bs, 1H), 3.32-3.23 (m, 1H), 2.95 (d, J=11.5 Hz, 2H), 2.83-2.79 (m, 2H), 2.63-2.59 (m, 2H), 2.48 (t, J=7.7 Hz, 2H), 2.29 (t, J=7.6 Hz, 2H), 2.23-2.16 (m, 2H), 2.07 (d, J=12.1 Hz, 2H), 1.64 (p, J=7.6 Hz, 2H), 1.57 (p, J=7.7 Hz, 2H), 1.53-1.45 (m, 2H), 1.38-1.30 (m, 2H); ¹³C NMR (126 MHz, Chloroform-d) δ 174.24, 145.05, 140.40, 131.27, 129.15, 128.68, 128.38, 126.02, 113.38, 60.67, 52.50, 51.43, 50.19, 34.73, 34.04, 33.91, 32.66, 31.38, 28.72, 24.83.

### Methyl 6-(4-(N-(1-phenethylpiperidin-4-yl)propionamido)phenyl)hexanoate

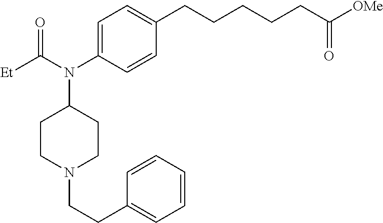 [SMILES: CCC(=O)N(c1ccc(CCCCCC(=O)OC)cc1)C1CCN(CCc2ccccc2)CC1]

To a solution of methyl 6-(4-((1-phenethylpiperidin-4-yl)amino)phenyl)hexanoate (470.0 mg, 1.15 mmol) in methylene chloride (12 mL) was added diisopropylethylamine (DIPEA, 0.4 mL, 2.3 mmol, 2.0 equiv.). The solution was cooled to 0° C. and treated dropwise with propionyl chloride (0.2 mL, 2.3 mmol, 2.0 equiv.). The resulting mixture was stirred for 3 h at ambient temperature. The mixture was quenched with water and extracted with methylene chloride. The organic phase was washed with saturated NaHCO₃ aqueous solution, dried over anhydrous Na₂SO₄ and evaporated in vacuo. The crude mixture was purified by flash column chromatography to give the title compound as a light yellow oil (92% yield); ¹H NMR (500 MHz, Chloroform-d) δ 7.31-7.22 (m, 3H), 7.18-7.13 (m, 4H), 6.97 (d, J=8.1 Hz, 2H), 4.77-4.59 (m, 1H), 3.67 (s, 3H), 3.00 (d, J=11.3 Hz, 2H), 2.88-2.80 (m, 2H), 2.77-2.69 (m, 2H), 2.62 (t, J=7.7 Hz, 2H), 2.57-2.50 (m, 2H), 2.33 (t, J=7.5 Hz, 2H), 2.19-2.11 (m, 2H), 1.93 (q, J=7.4 Hz, 2H), 1.79 (d, J=11.8 Hz, 2H), 1.72-1.60 (m, 4H), 1.48-1.35 (m, 2H), 1.01 (t, J=7.4 Hz, 3H). ¹³C NMR (126 MHz, Chloroform-d) δ 174.15, 173.70, 142.77, 140.23, 136.36, 130.19, 129.16, 128.63, 128.38, 126.03, 60.55, 53.12, 52.03, 51.49, 35.28, 33.97, 33.85, 31.03, 30.54, 28.78, 28.45, 24.75, 9.64.

### 6-(4-(N-(1-phenethylpiperidin-4-yl)propionamido)phenyl)hexanoic acid (Analogue 2)

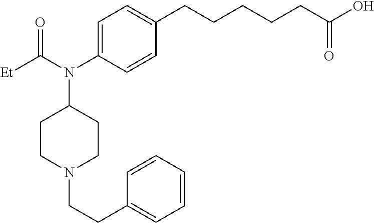 [SMILES: CCC(=O)N(c1ccc(CCCCCC(=O)O)cc1)C1CCN(CCc2ccccc2)CC1]

To a solution of methyl 6-(4-(N-(1-phenethylpiperidin-4-yl)propionamido)phenyl)hexanoate (162.6 mg, 0.35 mmol) in MeOH (1.8 mL) was added potassium hydroxide (0.20 g, 3.5 mmol) at 0° C. After being stirred for 4 h, the mixture was concentrated, and the residue was poured into saturated NH₄Cl aqueous solution and extracted with methylene chloride. The extract was washed with water and then brine, dried over anhydrous Na₂SO₄, and concentrated. The crude mixture was purified by flash column chromatography to give title compound as a light yellow solid (99% yield); ¹H NMR (500 MHz, Chloroform-d) δ 14.46 (s, 1H), 7.31-7.17 (m, 5H), 7.13 (d, J=6.9 Hz, 2H), 6.96 (d, J=7.3 Hz, 2H), 4.84-4.77 (m, 1H), 3.25 (d, J=10.0 Hz, 2H), 2.81 (d, J=8.2 Hz, 2H), 2.74-2.65 (m, 4H), 2.32 (t, J=11.4 Hz, 2H), 2.14-2.02 (m, 4H), 1.77 (d, J=11.4 Hz, 2H), 1.68-1.60 (m, 2H), 1.54-1.46 (m, 2H), 1.43-1.37 (m, 2H), 1.15-1.09 (m, 2H), 1.04 (t, J=7.1 Hz, 3H); ¹³C NMR (126 MHz, Chloroform-d) δ 177.31, 173.64, 143.31, 138.70, 135.41, 130.21, 129.60, 128.62, 128.51, 126.38, 58.96, 51.98, 50.14, 35.11, 34.88, 31.72, 30.31, 28.77, 28.50, 27.31, 25.18, 9.63.

### Example 2

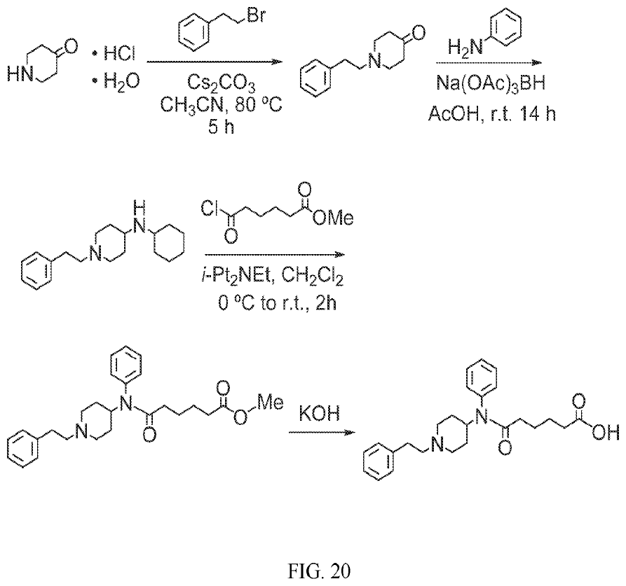

This example demonstrates a chemical synthesis of 6-oxo-6-((1-phenethylpiperidin-4-yl)(phenyl)amino)hexanoic acid (Analogue 3). FIG. 20 provides a schematic illustration of the chemical synthesis.

### 1-phenethylpiperidin-4-one

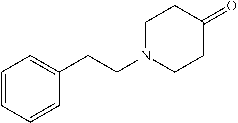 [SMILES: O=C1CCN(CCc2ccccc2)CC1]

To a solution of 4-piperidone monohydrate hydrochloride (5 g, 32.5 mmol) and CsCO₃ (23.3 g, 71.6 mmol) in acetonitrile (100 mL) was added 2-bromoethylbenzene (4.43 mL, 32.5 mmol), the mixture was stirred overnight at reflux. The solution was cooled to room temperature and quenched with water. The organic phase was dried with Na₂SO₄. The crude mixture was purified via column chromatography to give title compound as a white powder (3.4 g, 51% yield). ¹H NMR (500 MHz, CDCl₃) δ 7.33-7.28 (m, 2H), 7.24-7.19 (m, 3H), 2.90-2.80 (m, 6H), 2.78-2.70 (m, 2H), 2.49 (t, J=6.1 Hz, 4H); ¹³C NMR (126 MHz, CDCl₃) δ 140.11, 128.81, 128.61, 126.35, 59.44, 53.23, 41.39, 34.28; ¹³C NMR (126 MHz, CDCl₃) δ 140.11, 128.81, 128.61, 126.35, 59.44, 53.23, 41.39, 34.28.

### 1-phenethyl-N-phenylpiperidin-4-amine

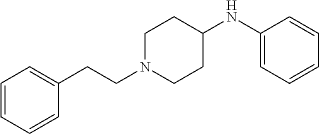 [SMILES: c1ccc(CCN2CCC(Nc3ccccc3)CC2)cc1]

To a solution of aniline (98 mg, 1.05 mmol) in methylene chloride (7.5 mL) at 0° C. was added acetic acid (0.01 mL, 1.05 mmol). To the mixture, N-phenylethylpiperidin-4-one (214 mg, 1.05 mmol) was added as a solution in methylene chloride (3.0 mL), followed by the portionwise addition of sodium triacetoxyborohydride (335.0 mg, 1.58 mmol, 1.1 equiv.). The reaction mixture was stirred at ambient temperature for 14 h. After this time, methanol was added to the mixture and all contents transferred to a separatory funnel. The mixture was partitioned (CH₂Cl₂/saturated NaHCO₃). Once neutralized, the organic phase was washed with brine (NaCl/H₂O), dried over Na₂SO₄ and concentrated in vacuo. The crude mixture was purified by flash column chromatography to give title compound as a light yellow oil. (188 mg, 64% yield): ¹H NMR (500 MHz, CDCl₃) δ 7.33-7.27 (m, 2H), 7.22 (d, J=7.0 Hz, 3H), 7.20-7.15 (m, 2H), 6.69 (tt, J=7.2, 1.1 Hz, 1H), 6.64-6.59 (m, 2H), 3.59-3.46 (m, 1H), 3.33 (tt, J=9.7, 4.0 Hz, 1H), 2.98 (dd, J=11.1, 4.7 Hz, 2H), 2.87-2.79 (m, 2H), 2.67-2.59 (m, 2H), 2.22 (td, J=11.4, 2.6 Hz, 2H), 2.14-2.06 (m, 2H), 1.52 (dtd, J=13.7, 10.5, 3.6 Hz, 2H); ¹³C NMR (126 MHz, CDCl₃) δ 147.21, 140.50, 129.45, 128.83, 128.53, 126.19, 117.35, 113.38, 60.78, 52.61, 50.04, 34.02, 32.69.

### Methyl 6-oxo-6-((1-phenethylpiperidin-4-yl)(phenyl)amino)hexanoate

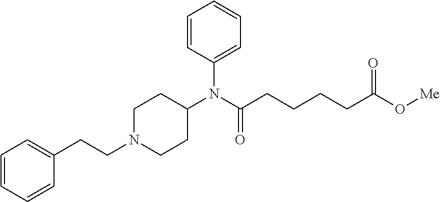 [SMILES: COC(=O)CCCCC(=O)N(c1ccccc1)C1CCN(CCc2ccccc2)CC1]

1-phenethyl-N-phenylpiperidin-4-amine (280.4 mg, 1.0 mmol) was dissolved in methylene chloride (20 mL) in a 100 mL round bottom flask equipped with a small stir bar and was treated with diisopropylethylamine (DIPEA, 0.35 mL, 2.0 mmol, 2.0 equiv.). The solution was cooled with an ice bath and treated dropwise with methyl adipoyl chloride (0.17 mL, 1.1 mmol). The resulting mixture was stirred for 2 h at ambient temperature. The mixture was transferred to a separatory funnel and partitioned (CH₂Cl₂/H₂O). The organic phase was washed with brine, saturated NaHCO₃, dried over anhydrous Na₂SO₄ and evaporated in vacuo. The crude mixture was purified by flash column chromatography to give title compound as a light yellow oil (92%); ¹H NMR (500 MHz, Chloroform-d) δ 7.41-7.34 (m, 3H), 7.28-7.22 (m, 3H), 7.17-7.13 (m, 2H), 7.07 (d, J=7.2 Hz, 2H), 4.67 (tt, J=12.1, 3.6 Hz, 1H), 3.62 (s, 3H), 2.99 (d, J=11.5 Hz, 2H), 2.74-2.70 (m, 2H), 2.56-2.50 (m, 2H), 2.22 (t, J=7.3 Hz, 2H), 2.18-2.13 (m, 2H), 1.92 (t, J=7.2 Hz, 2H), 1.80 (d, J=12.0 Hz, 2H), 1.61-1.55 (m, 2H), 1.54-1.47 (m, 2H), 1.43 (qd, J=12.3, 3.4 Hz, 2H); ¹³C NMR (126 MHz, Chloroform-d) δ 173.88, 172.18, 140.24, 138.72, 130.42, 129.33, 128.62, 128.36, 128.34, 126.01, 60.50, 53.09, 52.19, 51.44, 34.68, 33.86, 33.80, 30.58, 24.87, 24.50.

### 6-oxo-6-((1-phenethylpiperidin-4-yl)(phenyl)amino)hexanoic acid (Analogue 3)

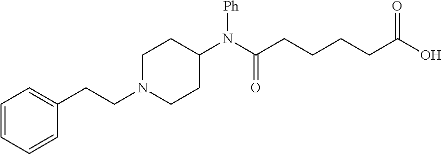 [SMILES: O=C(O)CCCCC(=O)N(c1ccccc1)C1CCN(CCc2ccccc2)CC1]

To a solution of methyl 6-oxo-6-((1-phenethylpiperidin-4-yl)(phenyl)amino)hexanoate (169.0 mg, 0.4 mmol) in MeOH (2.0 mL) was added potassium hydroxide (0.22 g, 4.0 mmol) at 0° C. After being stirred for 4 h, the mixture was concentrated, and the residue was poured into saturated NH₄Cl aqueous solution, and extracted with dichloromethane. The extract was washed with water and then brine, dried over anhydrous MgSO₄, and concentrated. The crude mixture was purified by flash column chromatography to give title compound as a light yellow solid (99%); ¹H NMR (500 MHz, Chloroform-d) δ 12.44 (s, 1H), 7.37 (t, J=7.4 Hz, 2H), 7.33-7.29 (m, 1H), 7.27-7.23 (m, 2H), 7.19 (t, J=7.3 Hz, 1H), 7.17-7.13 (m, 2H), 7.06 (d, J=7.3 Hz, 2H), 4.77-4.69 (m, 1H), 3.34 (d, J=10.5 Hz, 2H), 2.95-2.80 (m, 4H), 2.51 (t, J=11.2 Hz, 2H), 2.05 (t, J=7.6 Hz, 2H), 1.93 (t, J=7.5 Hz, 2H), 1.85 (d, J=11.8 Hz, 2H), 1.76-1.65 (m, 2H), 1.53 (p, J=7.6 Hz, 2H), 1.37 (p, J=7.7 Hz, 2H); ¹³C NMR (126 MHz, Chloroform-d) δ 177.32, 172.79, 138.12, 1³7.91, 130.02, 129.58, 128.73, 128.66, 128.64, 126.65, 58.62, 52.06, 50.75, 34.80, 31.45, 28.42, 24.97, 24.88.

### Example 3

This example demonstrates a chemical synthesis of 6-(4-(2-(4-(2-oxo-3,4-dihydroquinolin-1(2H)-yl)piperidin-1-yl)ethyl)phenyl)hexanoic acid (analogue 4).

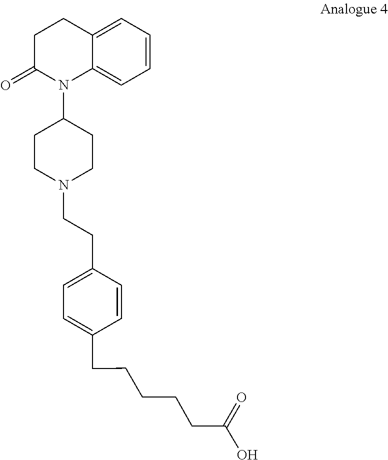 [SMILES: O=C(O)CCCCCc1ccc(CCN2CCC(N3C(=O)CCc4ccccc43)CC2)cc1]

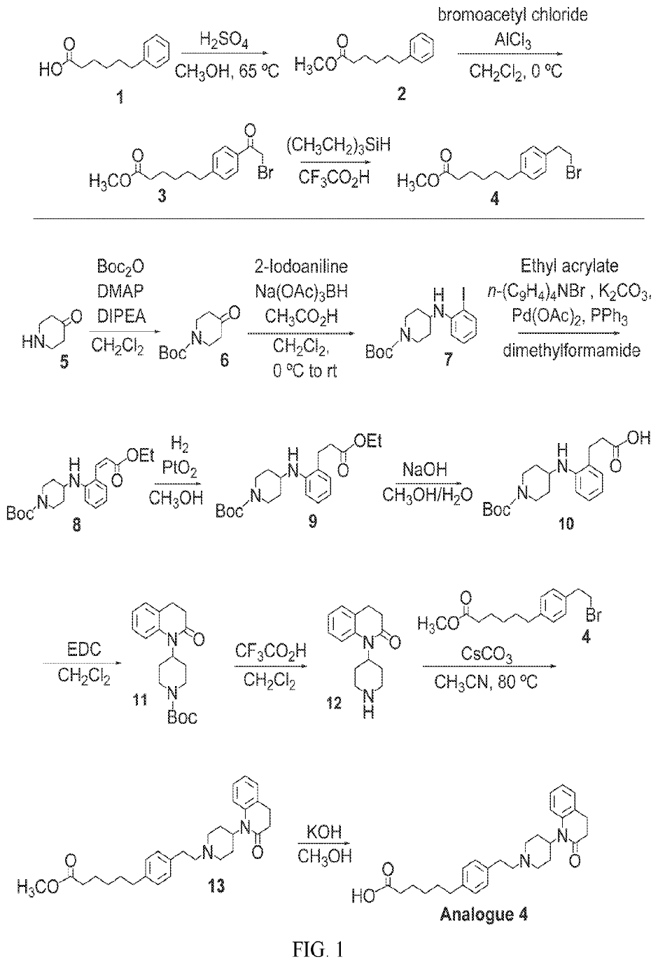

6-Phenylhexanoic acid (1) was converted to the corresponding methyl ester 2 by treatment with sulfuric acid in methanol. Subsequent Friedel-Crafts acylation with bromoacetyl chloride and aluminum(III) chloride furnished the bromoketone 3, which was then reductively deoxygenated with triethylsilane and trifluoroacetic acid (TFA) to produce bromide 4. Meanwhile, 4-piperidone (5) was protected as the N-Boc carbamate 6 using di-tert-butyl decarbonate (Boc₂)O in the presence of dimethylaminopyridine (DMAP) and diisopropylethylamine (DIPEA). Reductive amination of 6 with 2-iodoaniline using sodium triacetoxyborane and trifluoroacetic acid the furnished compound 7. Heck coupling of 7 with ethyl acrylate was achieved using palladium(II) acetate and triphenylphosphine in the presence of tetrabutylammonium bromide and potassium carbonate in dimethylformamide, leading the unsaturated ester 8. This compound was hydrogenated over platinum oxide in methanol to produce 9 and then saponified with sodium hydroxide in methanol/water to give rise to the acid 10. Treatment of 10 with EDC in methylene chloride solvent effected lactamization to produce 11, which could be deprotected with trifluoroacetic acid to provide amine 12. Reaction of 12 with bromide 4 in the presence of cesium(II) carbonate in acetonitrile at elevated temperature led to compound 13. Finally, saponification with potassium hydroxide in methanol furnished analogue 4. The chemical scheme of the synthetic method is set forth in FIG. 1.

### Example 4

This example demonstrates a chemical synthesis of 6-(2-oxo-1-(1-phenethylpiperidin-4-yl)-1,2,3,4-tetrahydroquinolin-6-yl)hexanoic acid (analogue 5).

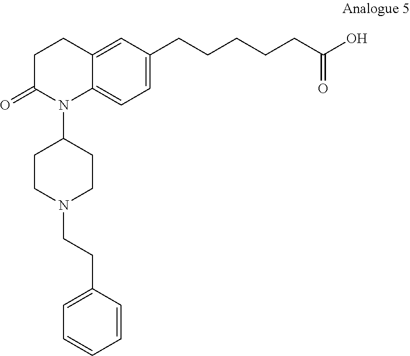 [SMILES: O=C(O)CCCCCc1ccc2c(c1)CCC(=O)N2C1CCN(CCc2ccccc2)CC1]

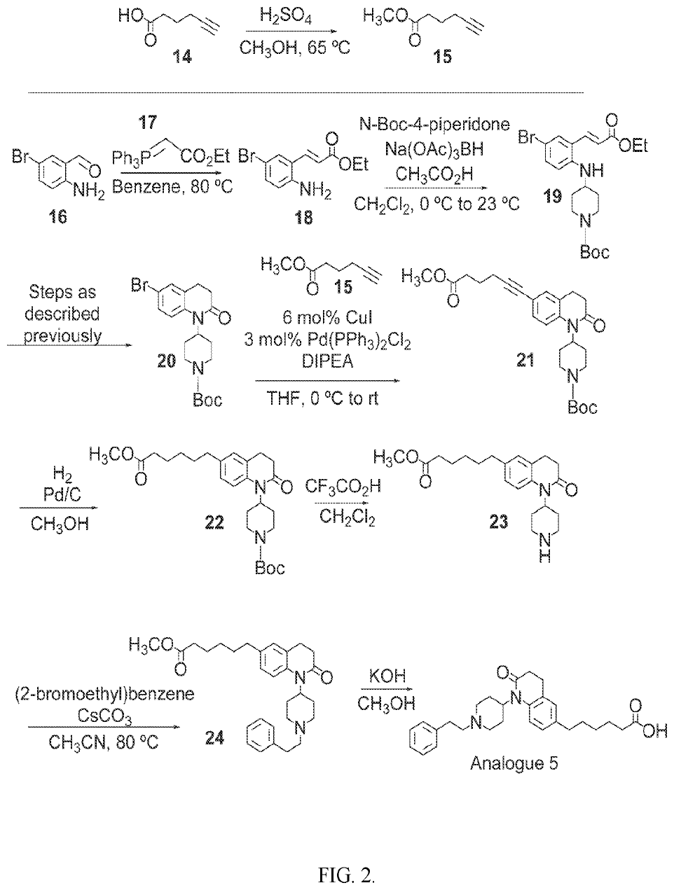

5-Hexynoic acid (14) was treated with sulfuric acid in methanol at 65° C., which furnished the methyl 5-hexynoate (15). Meanwhile, aldehyde 16 was reacted with ylide 17 to produce unsaturated ester 18. Reductive amination with N-Boc-4-piperidone using sodium triacetoxyborohydride and acetic acid gave rise to compound 19, which was converted to 20 by the same sequence of steps used to convert 8 to 11 described above for the synthesis of analogue 4. Sonogashira coupling of 20 with alkyne 15 then led to adduct 21. Hydrogenation of the triple bond followed by removal of the Boc protecting group with trifluoroacetic acid provided amine 23. Alkylation of this compound with (2-bromoethyl)benzene with cesium(II) carbonate as base at elevated temperature furnished compound 24. Finally, saponification with potassium hydroxide yielded analogue 5. The chemical scheme of the synthetic method is set forth in FIG. 2.

### Example 5

This example demonstrates a chemical synthesis of 6-(2-oxo-1-(1-phenethylpiperidin-4-yl)-1,2,3,4-tetrahydroquinolin-3-yl)hexanoic acid (analogue 6) in an aspect of the invention.

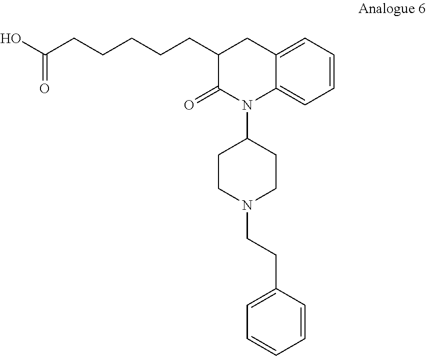 [SMILES: O=C(O)CCCCCC1Cc2ccccc2N(C2CCN(CCc3ccccc3)CC2)C1=O]

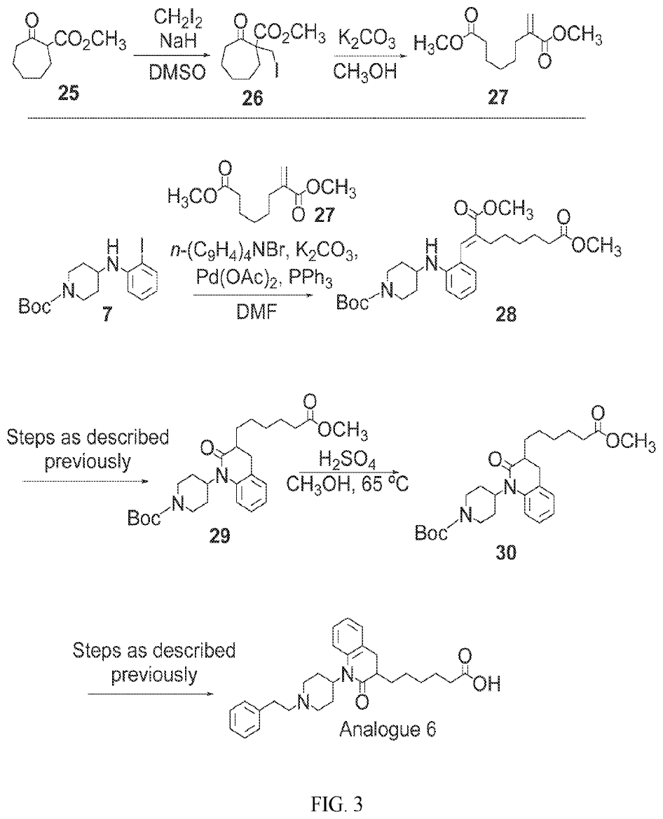

Ketoester 25 was alkylated with diiodomethane using sodium hydride in dimethylsulfoxide to produce 26. Subsequent fragmentation with potassium carbonate in methanol then furnished diester 27. This compound was then subjected to Heck reaction with aryl iodide 7 (prepared as described for analogue 4) using the same conditions as described for the conversion of 7 to 8, which led to the generation of compound 28. This compound was converted to lactam 29 using the same sequence of steps used to convert 8 to 11 described above for the synthesis of analogue 4. Esterification of 29 to produce 30 was accomplished using sulfuric acid in methanol with heating. Finally, compound 30 was converted to analogue 6 using the same sequence of steps used to convert compound 22 to analogue 6 (i.e., Boc removal, alkylation, saponification). The chemical scheme of the synthetic method is set forth in FIG. 3.

### Example 6

This example demonstrates a chemical synthesis of 6-(4-(N-(1,3,4,6,7,11b-hexahydro-2H-pyrido[2,1-a]isoquinolin-2-yl)propionamido)phenyl)hexanoic acid (analogue 7) in an aspect of the invention.

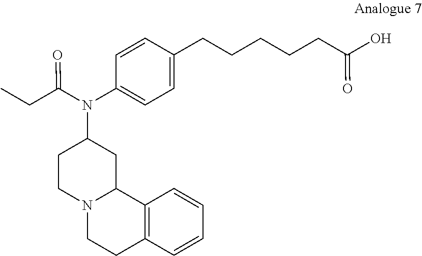 [SMILES: CCC(=O)N(c1ccc(CCCCCC(=O)O)cc1)C1CCN2CCc3ccccc3C2C1]

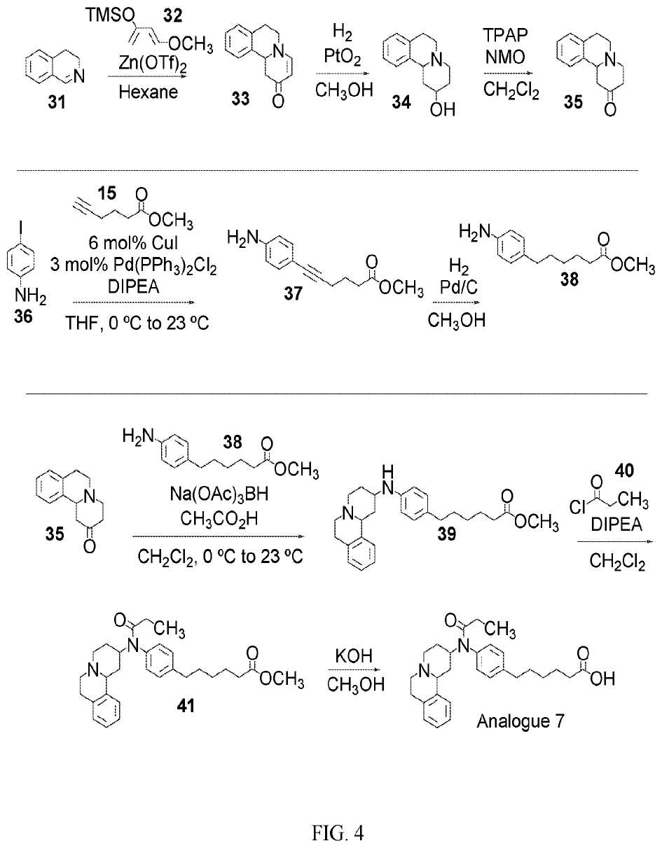

Dihydroisoquinoline 31 was reacted with Danishefsky's diene 32 in the presence of zinc(II)trifluoromethanesulfonate to furnish compound 33. Hydrogenation over platinum oxide in methanol then led to alcohol 34, which was oxidized to ketone 35 using tetrapropylammonium perruthenate and N-methylmorpholine N-oxide. Meanwhile, 4-iodoaniline (36) was subjected to Sonogashira coupling with alkyne 15 using catalytic copper(I) iodide and palladium(II)bis(triphenylphosphine)dichloride to yield compound 37. Hydrogenation over palladium on carbon then furnished aniline 38. This compound was then used in a reductive amination with ketone 35 using sodium triacetoxyborohydride and acetic acid to produce compound 39. Acylation of 39 with propionyl chloride (40) in the presence of diisopropylethylamine gave rise to 41, which was saponified using potassium hydroxide to furnish analogue 7. The chemical scheme of the synthetic method is set forth in FIG. 4.

### Example 7

This example demonstrates a chemical synthesis of 7-((1,3,4,6,7,11b-hexahydro-2H-pyrido[2,1-a]isoquinolin-2-yl)(phenyl)amino)-7-oxoheptanoic acid (analogue 8) in an aspect of the invention.

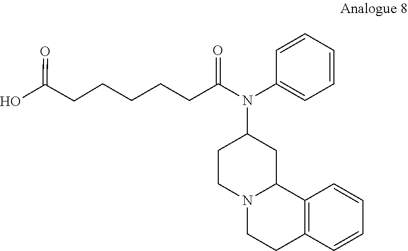 [SMILES: O=C(O)CCCCCC(=O)N(c1ccccc1)C1CCN2CCc3ccccc3C2C1]

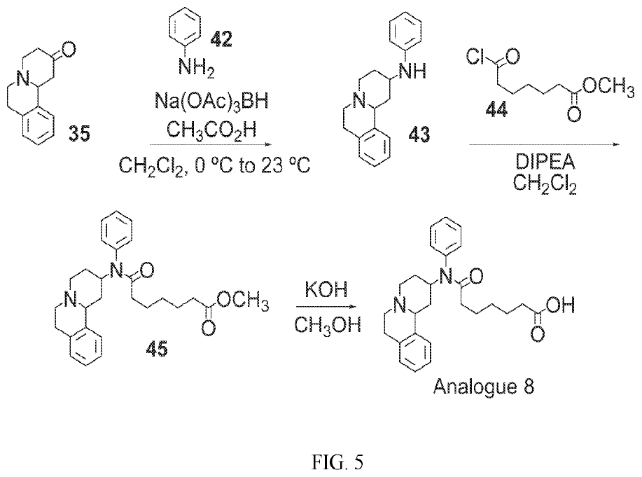

Ketone 35, synthesized as described above for analogue 7, was subjected to reductive amination with aniline (42) using sodium triacetoxyborohydride and acetic acid to furnish compound 43. This material was acylated with acid chloride 44 in the presence of diisopropylethylamine to produce ester 45, which was saponified with potassium hydroxide in methanol to yield analogue 8. The chemical scheme of the synthetic method is set forth in FIG. 5.

### Example 8

This example demonstrates a chemical synthesis of 6-(4-(2-(2-oxo-1-phenyloctahydro-1,6-naphthyridin-6(2H)-yl)ethyl)phenyl)hexanoic acid (analogue 9) in an aspect of the invention.

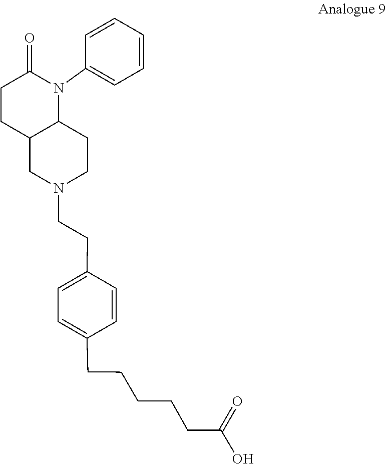 [SMILES: O=C(O)CCCCCc1ccc(CCN2CCC3C(CCC(=O)N3c3ccccc3)C2)cc1]

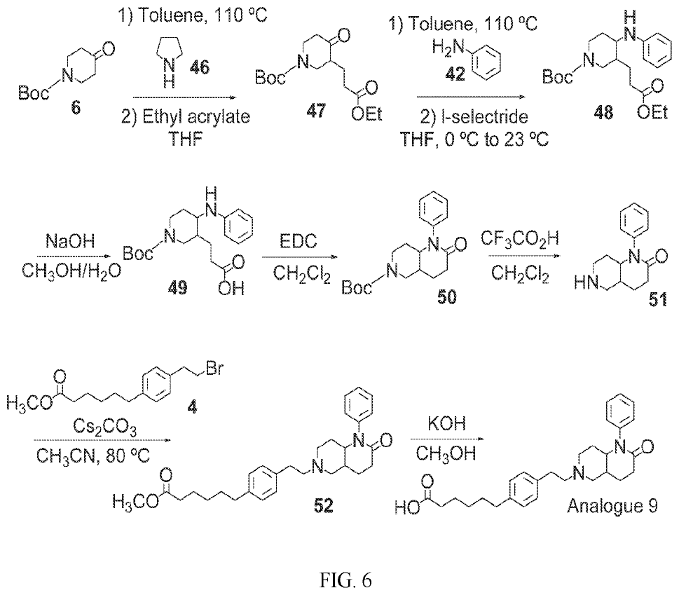

Ketone 6, synthesized as described above for analogue 4, was alkylated by condensation with pyrrolidine (46) at elevated temperature, followed by reaction with ethylacrylate, leading to the formation of ester 47. This compound was subjected to reductive amination with aniline (42) using 1-selectride to furnish compound 48. Saponification with sodium hydroxide in methanol and water led to acid 49, which was lactamized to 50 and deprotected to form 51 using steps as previously described. Alklyation of 51 with bromide 4, synthesized as described above for analogue 4, using cesium carbonate at elevated temperature, followed by saponification of the resulting compound 52 using potassium hydroxide in methanol then furnished analogue 9. The chemical scheme of the synthetic method is set forth in FIG. 6.

### Example 9

This example demonstrates a chemical synthesis of 6-(4-(2-oxo-6-phenethyloctahydro-1,6-naphthyridin-1(2H)-yl)phenyl)hexanoic acid (analogue 10) in an aspect of the invention.

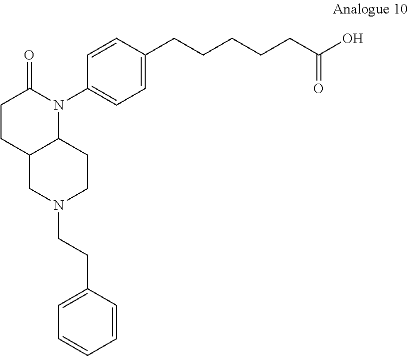 [SMILES: O=C(O)CCCCCc1ccc(N2C(=O)CCC3CN(CCc4ccccc4)CCC32)cc1]

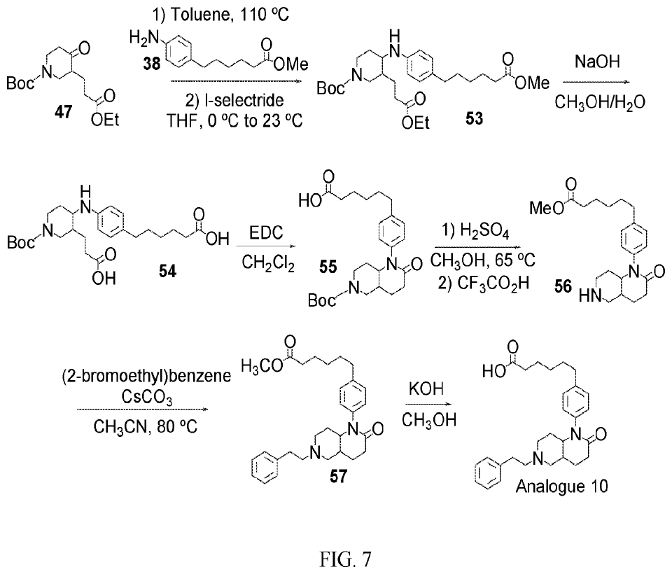

Ketone 47, prepared as described above for analogue 6, and aniline 38, prepared as described above for analogue 7, were subjected to reductive amination by first condensation in toluene at elevated temperature followed by reduction with 1-selectride to furnish compound 53. Global saponification was achieved using sodium hydroxide in methanol and water, leading to the diacid 54. Lactamization using EDC gave rise to lactam 55, which was converted to compound 56 by treatment with sulfuric acid in methanol followed by trifluoroacetic acid to ensure removal of the BOC protecting group. Alkylation of 56 with (2-bromoethyl)benzene in the presence of cesium carbonate at elevated temperature furnished the ester 57, which was then saponified with potassium hydroxide in methanol to provide analogue 10. The chemical scheme of the synthetic method is set forth in FIG. 7.

### Example 10

This example demonstrates a chemical synthesis of 5-(2-oxo-6-phenethyl-1-phenyldecahydro-1,6-naphthyridin-3-yl)pentanoic acid (analogue 11) in an aspect of the invention.

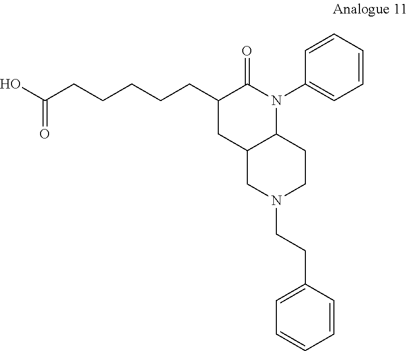 [SMILES: O=C(O)CCCCCC1CC2CN(CCc3ccccc3)CCC2N(c2ccccc2)C1=O]

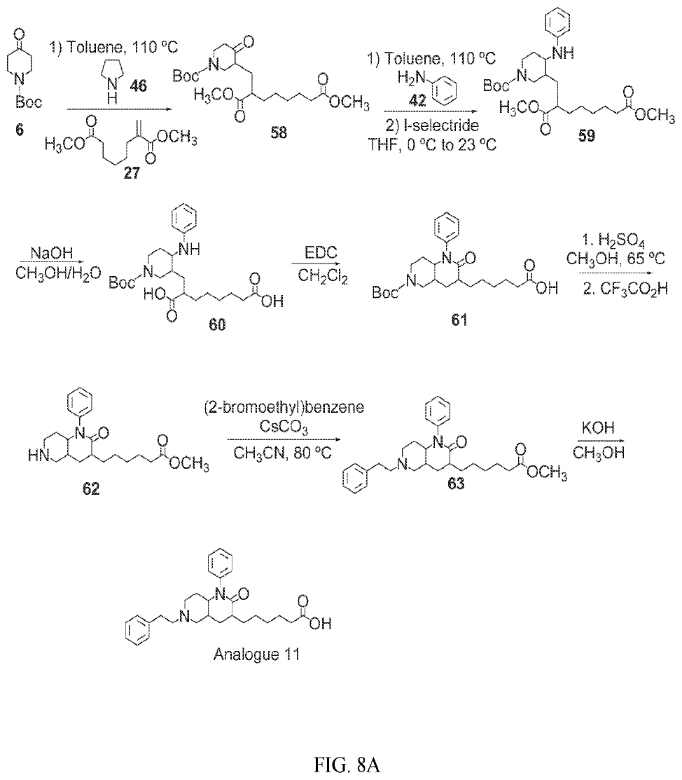

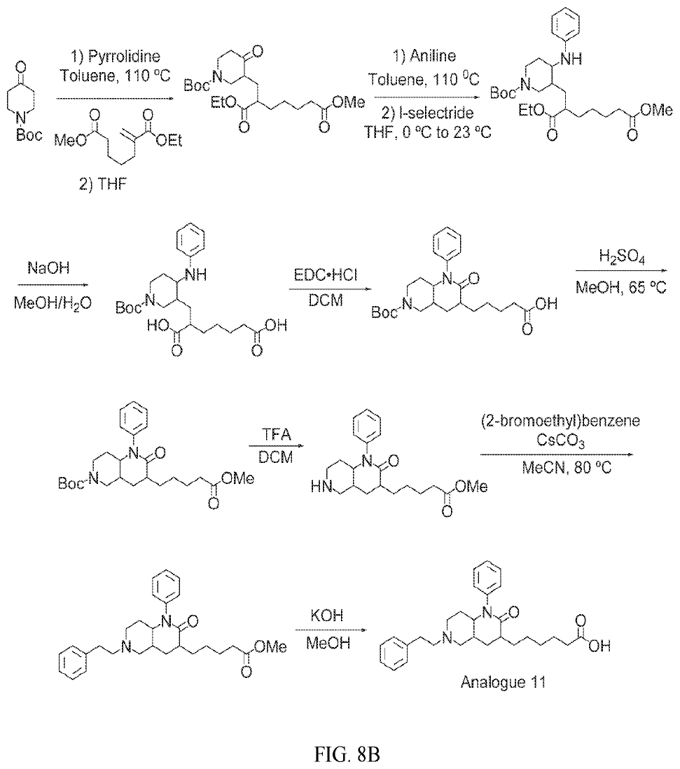

Ketone 6, prepared as described above for analogue 4, was condensed with pyrrolidine (46) and then reacted with diester 27, prepared as described above for analogue 6, which yielded compound 58. Reductive amination with aniline (42) using 1-selectride then furnished 59. This material was saponified with sodium hydroxide in methanol and water to produce diacid 60, which was then lactamized using EDC, leading to compound 61. Esterification by treatment with sulfuric acid in methanol followed by treatment with trifluoroacetic acid to ensure removal of the Boc protecting group then furnished the bicyclic amine 62. Alkylation of 57 with (2-bromoethyl)benzene in the presence of cesium carbonate led to compound 63. Finally, saponification with potassium hydroxide in methanol furnished analogue 11. The chemical scheme of the synthetic method is set forth in FIG. 8A. An alternative method is set forth in FIG. 8B.

### Example 11

The following example illustrates the preparation of conjugates comprising fentanyl analogues conjugated to adenoviral capsid proteins.

Detergent-disrupted human adenovirus type 5 (dAd5) was prepared by disrupting recombinant serotype 5 E1-E3—Ad vector (Ad5) expressing b-galactosidase with 0.5% sodium dodecyl sulfate (SDS) at 56° C. for 45 seconds.

Carfentanil (CF) was activated overnight at 4° C. in charging solution consisting of 1-ethyl-3-[3-dimethylaminopropyl] carbodiimide hydrochloride (EDC) and N-hydroxysulfosuccinimide (S-NHS). The disrupted Ad5 vector (dAd5) was then conjugated overnight at 4° C. to carfentanil to provide dAd5-Carfentanil conjugate (dAd5-CF). Western analysis with anti-fentanyl antibody confirmed conjugation of the hapten to capsid proteins including hexon, penton and fiber.

Conjugates 2 and 3 (also referred to as Haptens 2 and 3 or dAd5-Hapten 2 or dAd5-Hapten 3, respectively) were prepared from fentanyl analogues 2 and 3 by the same method as used to prepare dAd5-CF. Fentanyl analogues 2 and 3 had the following structures:

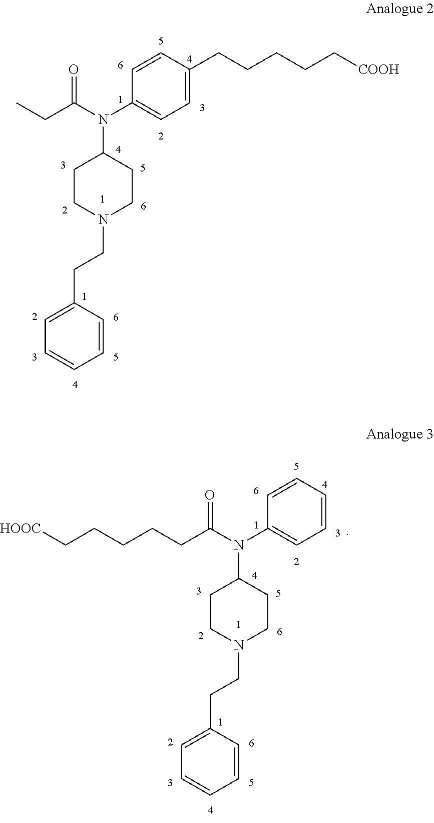 [SMILES: CCC(=O)N(c1ccc(CCCCCC(=O)O)cc1)C1CCN(CCc2ccccc2)CC1 ; O=C(O)CCCCCC(=O)N(c1ccccc1)C1CCN(CCc2ccccc2)CC1]

Each analogue was activated with EDC/NHS (N-ethyl-N′-(3-(dimethylamino)propyl) carbodiimide/N-hydroxysuccinimide) and combined with the dAd5 to provide the corresponding conjugate:

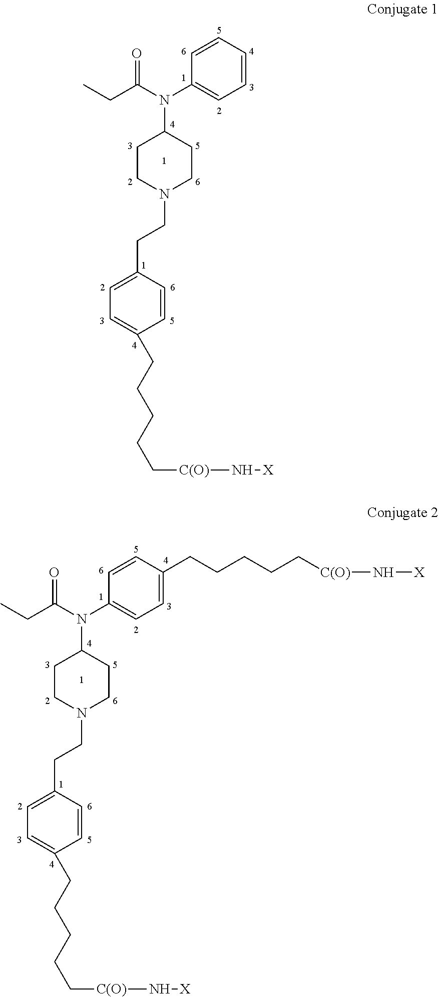

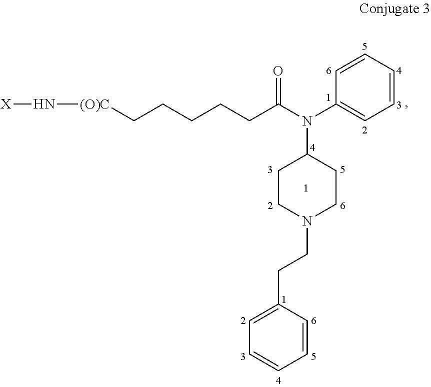

wherein X is an adenoviral capsid protein of the disrupted adenovirus (dAd5).

### Example 12

The following example illustrates the generation of anti-fentanyl antibodies in mice using the dAd5-CF conjugate provided herein.

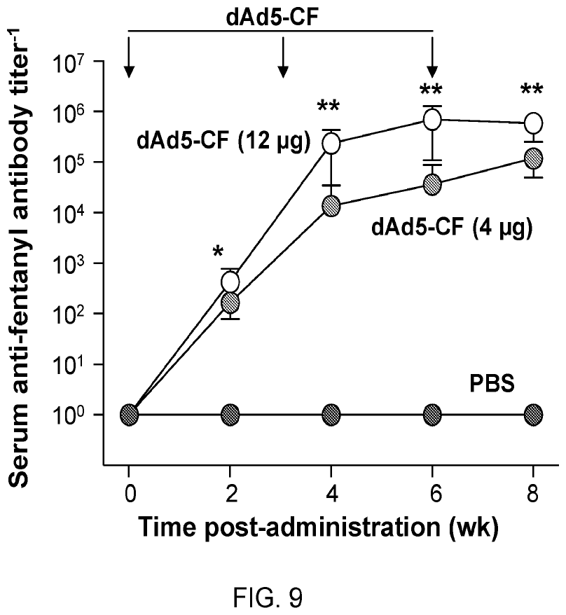

dAd5-CF was formulated with carbomer-lecithin adjuvant (AJDUPLEX™ (Advanced BioAdjuvants, LLC, Omaha, NE), and administered to Balb/c mice (n=10) at 4 μg and 12 μg dosages. The mice were given an initial dose (week 0) and booster doses at weeks 3 and 6 via intramuscular injection. Blood was collected biweekly from the transected tail vein, allowed to clot at 22° C., centrifuged at 1,000×g for 10 minutes at 22° C., and the resulting serum stored at −20° C. Assessment of serum anti-fentanyl antibody titers by enzyme-linked immunosorbent assay (ELISA) on BSA-carfentanil coated plate (FIG. 9) showed that at 4 weeks and later, both 4 μg and 12 μg doses produced higher serum anti-fentanyl antibodies than PBS-control (p<0.05). The 12 μg dose produced significantly higher serum anti-fentanyl antibody titers compared to the 4 μg dose, specifically at 4-8 week time points (p<0.05).

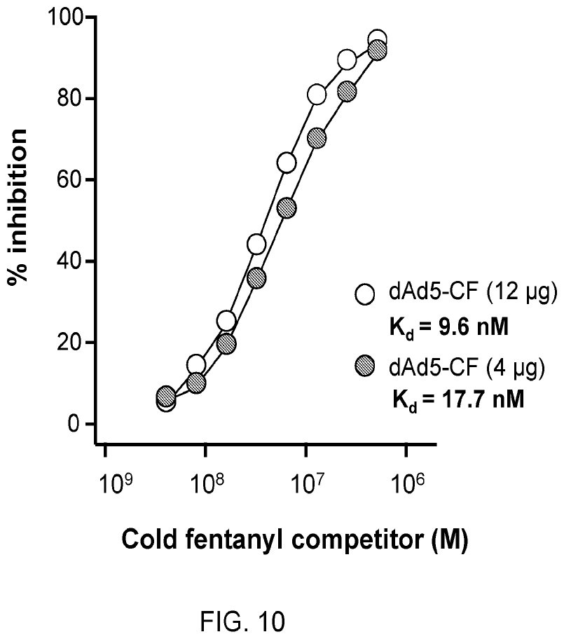

dAd5-CF (4 μg and 12 μg) immunized 8 wk mice sera were assayed for anti-fentanyl antibody affinity (Kd) using competitive radioimmunoassay. Pooled serum was incubated with 3H-fentanyl (0.1 μCi) in the presence of increasing concentrations of cold competitor, non-radiolabeled fentanyl. The percent inhibition of 3H-fentanyl binding was plotted as a function of competitor concentration and the affinity to fentanyl was calculated from the resulting curve (FIG. 10). Both dAd5-CF doses exhibited nanomolar anti-fentanyl antibody affinity at the 8-week time point (Kd=17.7 nM and 9.59 nM for 4 μg and 12 μg doses, respectively).

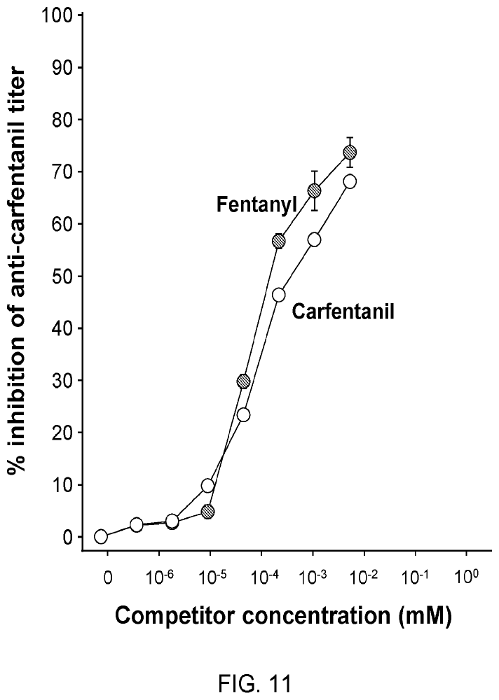

To determine specificity of the antibodies, binding of dAd5-CF immunized mouse sera to BSA-carfentanil coated ELISA plate was competed with fentanyl and carfentanil at increasing concentrations (FIG. 11). The dAd5-CF evoked antibodies recognized fentanyl and carfentanil with similar specificities.

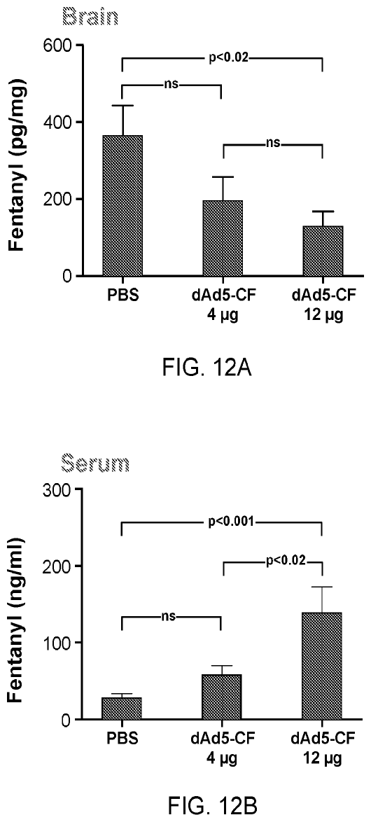

The effect of dAd5-CF on blood and brain concentration of fentanyl also was examined. Naive or dAd5-CF immunized mice were intravenously administered 1 μg of fentanyl containing 2 μCi 3H-fentanyl and one-minute post fentanyl administration, mice were decapitated, and brain and trunk blood collected separately. Brain tissue was homogenized in PBS, whereas trunk blood was centrifuged for 10 minutes (1000×g at 22° C.) to obtain serum. Samples were assayed for 3H (tritium) using a LS6500 Liquid Scintillation Counter and was serum results were normalized to serum volume (ng/mL), and brain results were normalized to total protein. For brain, one-way ANOVA revealed a significant main effect of dose (F2,25=3.945, p<0.04). Pairwise comparisons revealed the 12 μg (p<0.02), but not 4 μg dose (p=0.06), significantly reduced fentanyl concentration in brain relative to PBS-controls. For serum, one-way ANOVA revealed a significant main effect of dose (F2,25=7.736, p<0.003). Pairwise comparisons revealed the 12 μg dose yielded significantly higher fentanyl concentration in serum relative to both the 4 μg dose (p<0.02) and PBS-controls (p<0.001); however, the 4 μg dose and PBS-controls did not significantly differ (p=0.3). Results are shown in FIG. 12A and FIG. 12B.

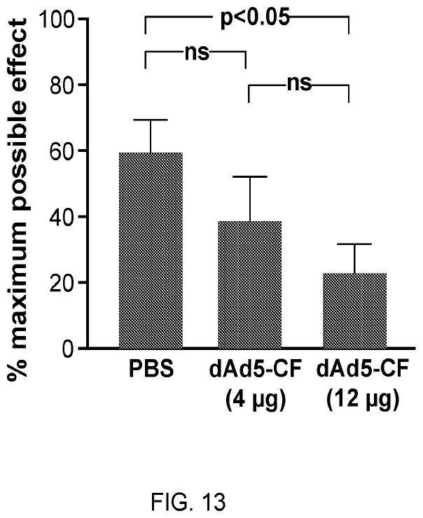

The immunized mice also were examined for antinocioception in the form of heat tolerance using a hot-plate latency test. Naive or dAd5-CF immunized mice were administered 1× PBS (5 mL/kg) via subcutaneous (s.c.) injection and 15 minutes later were placed on a hot-plate maintained at 55° C. Latency to observe a nociceptive response, defined as the time until a response of hind paw withdrawal and licking or jumping, was recorded with a stopwatch. In the absence of such a response, testing terminated at 35 seconds to avoid tissue damage. 15 minutes post-baseline testing, mice were administered fentanyl-HCl (0.05 mg/kg, s.c.), and 15 minutes later, were again placed on the hot-plate to measure latency to nociceptive response. The percentage maximum possible effect (% MPE) was calculated as follows. (fentanyl latency−PBS latency)/(cutoff time[35 seconds]−PBS latency)×100. One-way ANOVA revealed a significant main effect of dose (F2,26=3.478, p<0.05). Pairwise comparisons revealed the 12 μg dose (p<0.02), but not 4 μg (p=0.16), significantly reduced the % maximum possible effect compared to PBS-controls. Results are presented in FIG. 13.

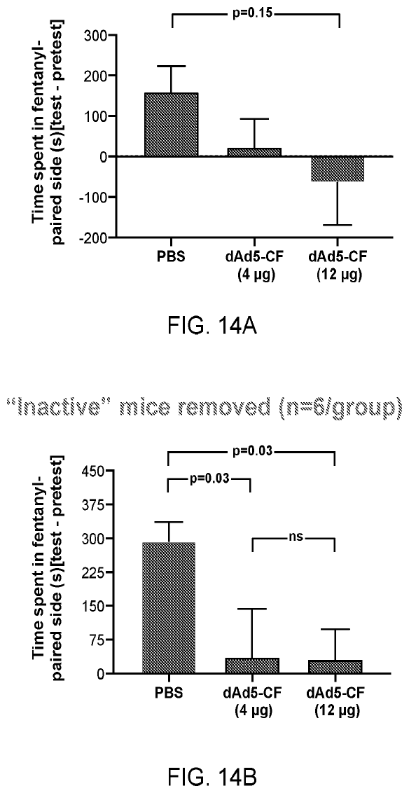

Conditioned Place Preference was evaluated at 9 wk post-vaccination. Fentanyl reward was given on 1 side of 2-sided chamber and the increase in time spent on fentanyl side over time spent same side prior to fentanyl, with and without vaccination were evaluated. Mice that spend 100% of time during pretests and test on same side were excluded. Results are presented in FIGS. 14A and 14B. The dAd5-CF vaccination removed the preference for time spent in fentanyl side of the chamber (p<0.04).

### Example 13

The following example illustrates the use of fentanyl analogues conjugated to dAd5 to generate anti-fentanyl antibodies in mice.

Conjugates 2 and 3 (also referred to as Haptens 2 and 3 or dAd5-Hapten 2 or dAd5-Hapten 3, respectively) were formulated with carbomer-lecithin adjuvant (AJDUPLEX™ (Advanced BioAdjuvants, LLC, Omaha, NE), and administered to Balb/c mice (n=5/group) at 2 μg, 4 μg, and 8 μg dosages. The mice were given an initial dose (week 0) and booster doses at weeks 3 and 6 via intramuscular injection.

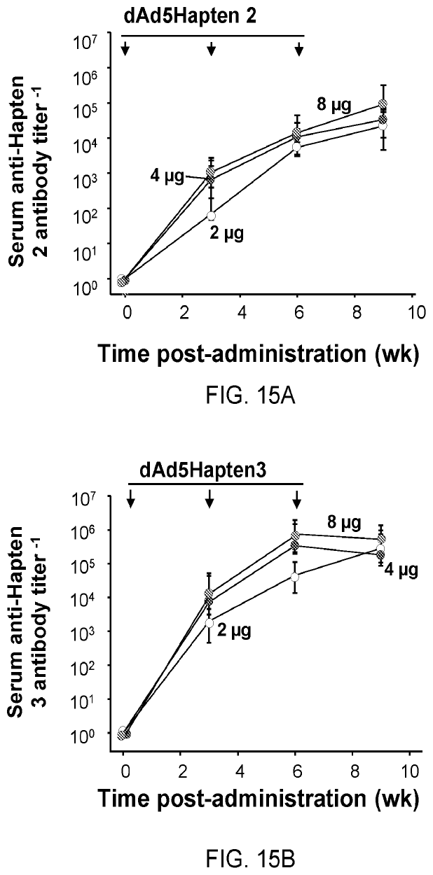

Serum anti-fentanyl antibody titers were evaluated on BSA-hapten 2 or BSA-hapten 3 coated plates by ELISA. Vaccine based on each hapten evoked high antibody titers. The results (FIGS. 15A and 15B) show that the fentanyl analogue conjugates produced significant anti-fentanyl antibody titers.

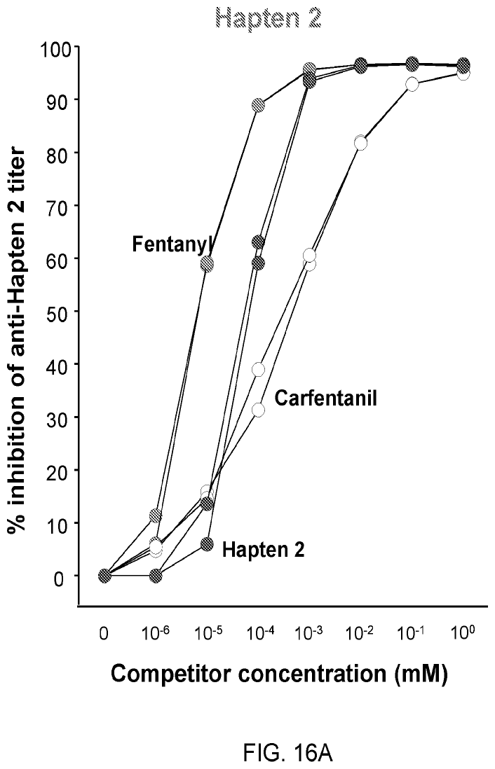

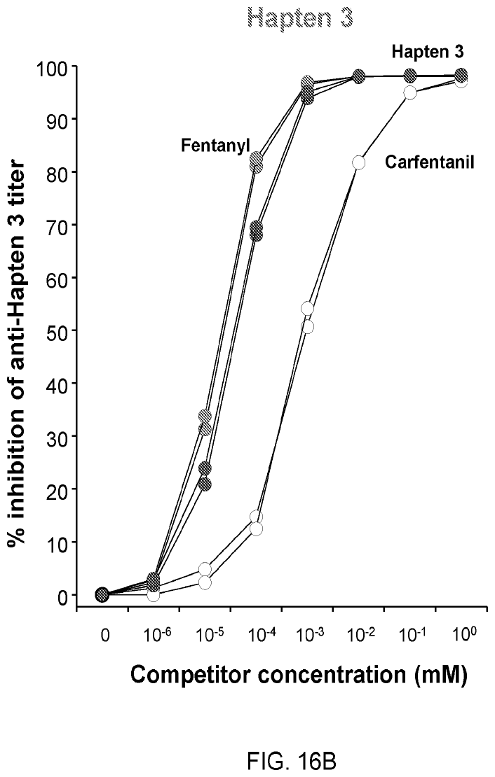

In an anti-fentanyl ELISA, binding of the mouse antisera to the plate was competed with fentanyl, carfentanil and hapten 2 or hapten 3. The results are provided in FIGS. 16A and 16B. Both hapten-based vaccines evoked antibodies with high fentanyl specificity.

### Example 14

The following example illustrates the effect of vaccination with fentanyl analogues conjugated to dAd5 in mice.

Conjugates 2 and 3 (also referred to as dAd5-Hapten 2 or dAd5-Hapten 3) were formulated with carbomer-lecithin adjuvant (AJDUPLEX™ (Advanced BioAdjuvants, LLC, Omaha, NE), and administered to C57BL/6 female mice (n=8-10/group) at 8 μg dosage. PBS served as a control. Mice were given an initial dose (week 0) and boost doses at weeks 3, 6, and 8.

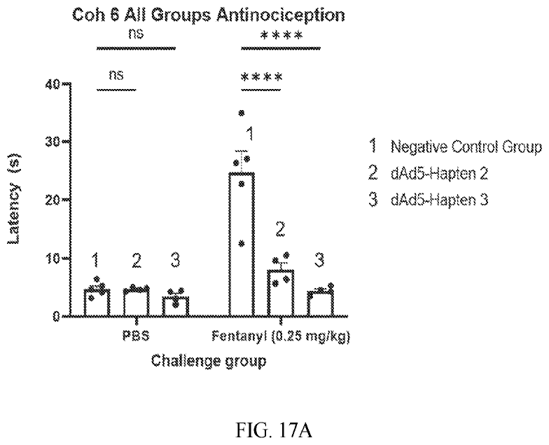

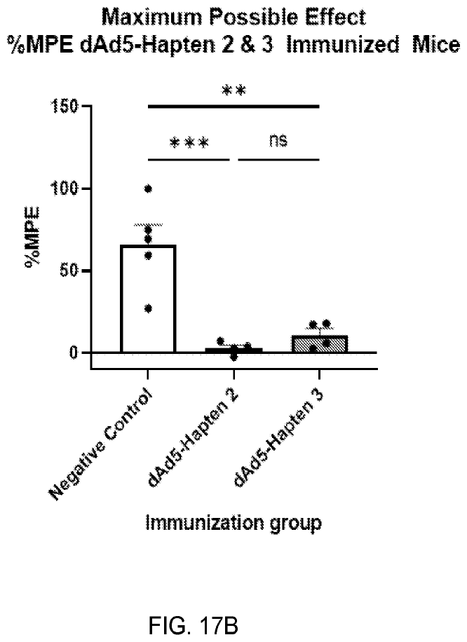

The effect of dAd5-Hapten 2 or dAd5-Hapten 3 immunization on antinociceptive response was analyzed by hot plate latency. Naive or dAd5-hapten 2 or dAd5-hapten 3 immunized mice were administered 1× PBS (5 mL/kg) via intraperitoneal route and 15 minutes later were placed on a hot-plate maintained at 55° C. Latency to observe a nociceptive response, defined as the time until a response of hind paw withdrawal and licking or jumping, was recorded with a stopwatch. In the absence of such a response, testing terminated at 35 seconds to avoid tissue damage. 15 minutes post-baseline testing, mice were administered fentanyl-HCl (0.25 mg/kg intraperitoneal), and 15 minutes later, were again placed on the hot-plate to measure latency to nociceptive response. The percentage maximum possible effect (% MPE) was calculated as follows: (fentanyl latency−PBS latency)/(cutoff time[35 seconds]−PBS latency)×100. The results are presented as seconds of latency (FIG. 17A) and percentage of maximum possible effect (FIG. 17B). Both dAd5-hapten 2 and dAd5-hapten 3 vaccination significantly reduced the % maximum possible effect compared to PBS-controls. Comparison of % MPE between dAd5-CF (prior example) and dAd5-hapten 2 or dAd5-hapten 3 immunized mice showed that dAd5-hapten 2 and dAd5-hapten 3 restored heat sensitivity better than dAd5-CF.

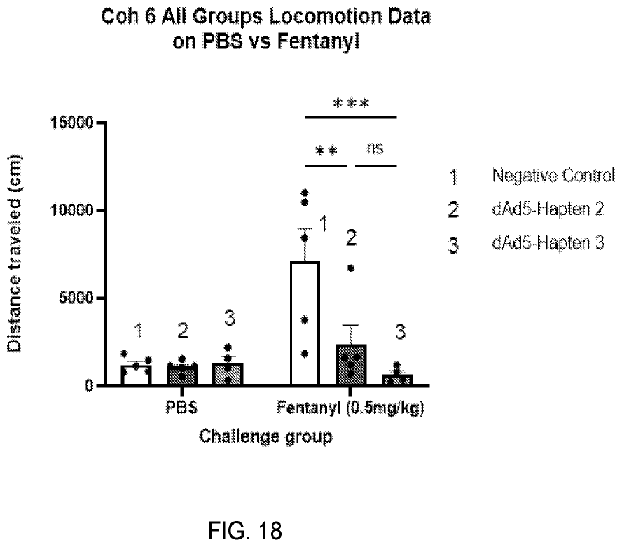

Fentanyl-induced hyperlocomotion by tracking distance in an open field for a specified period was measured. Total distance traveled by vaccinated mice compared to PBS administered mice following fentanyl (0.5 mg/kg, intraperitoneal) challenge was assessed. The results are presented in FIG. 18. Comparison of dAd5-hapten 2 or dAd5-hapten 3 vaccinated mice vs PBS control mice showed significant reduction of hyperlocomotive activity by the vaccines.

A lethal dose challenge was performed on the dAd5-Hapten 3 treated mice. Control or vaccinated mice were challenged with high dose fentanyl (15 mg/kg, tail vein injection). The survival of vaccinated mice was higher (78% for dAd5-hapten 2 and 80% for dAd5hapten 3) compared to the control mice (44%). Rapid death in control mice was observed, while most vaccinated mice survived over the longest measured time period, 2 hours post fentanyl challenge observation. No further mice mortality was observed after 2 hours. Based on the results, dAd5-Hapten 3 protected against lethal fentanyl challenge.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred aspects of this invention are described herein, including the best mode known to the inventors for carrying out the invention. Variations of those preferred aspects may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A fentanyl analogue having the structure of any of Formulae (1)-(7): 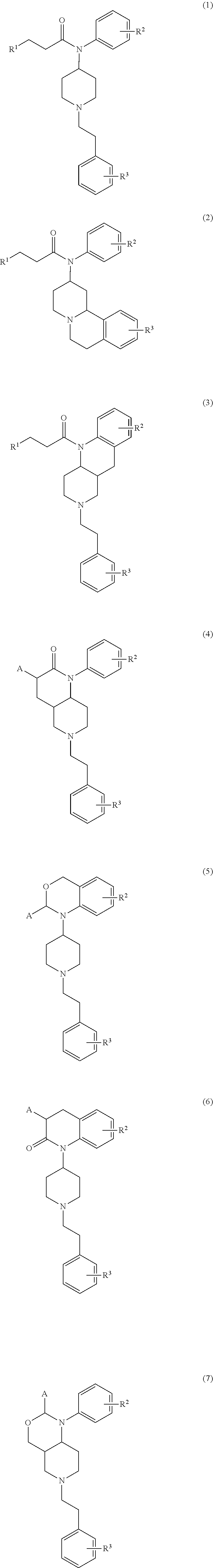
wherein
A is H or -alkyl-R¹;
R¹, R², and R³ are each independently H or —(C₁-C₁₂ alkyl)-COOH, provided that at least one of R¹, R², and R³ is —(C₁-C₁₂ alkyl)-COOH.

2. The fentanyl analogue of claim 1, having the following structure:

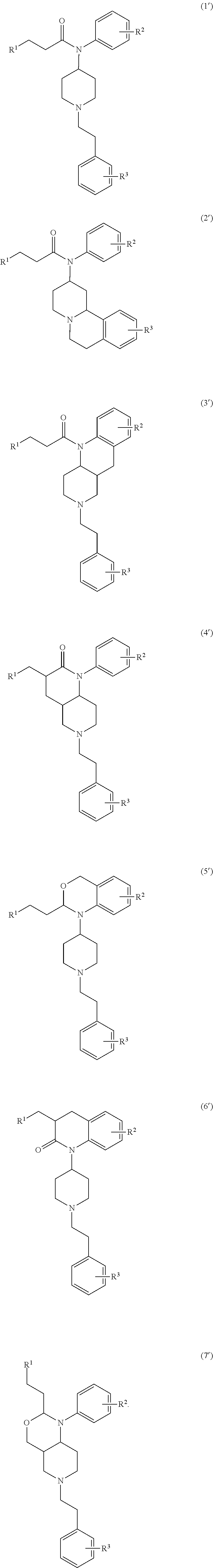

3. The fentanyl analogue of claim 1, wherein the fentanyl analogue is any one of Formulae (8)-(12): 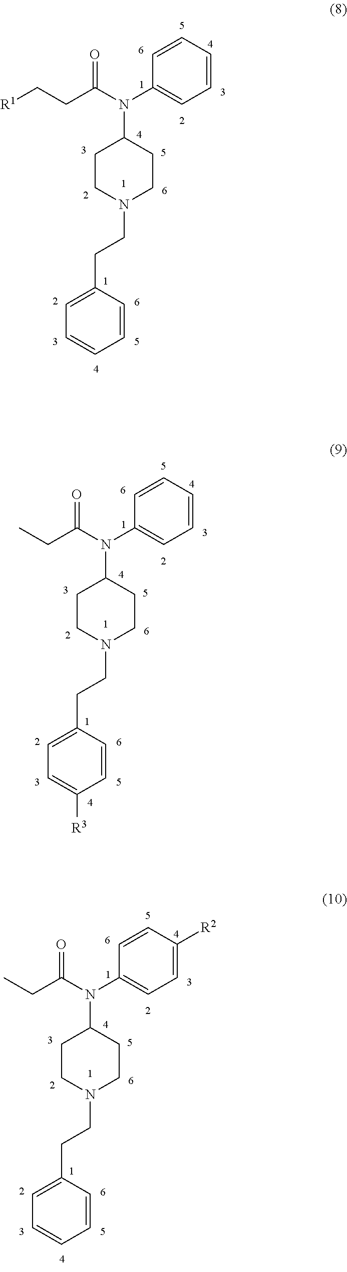
wherein R¹, R², and R³ are each —(C₁-C₁₂ alkyl)-COOH.

4. The fentanyl analogue of claim 1 having the following structure:

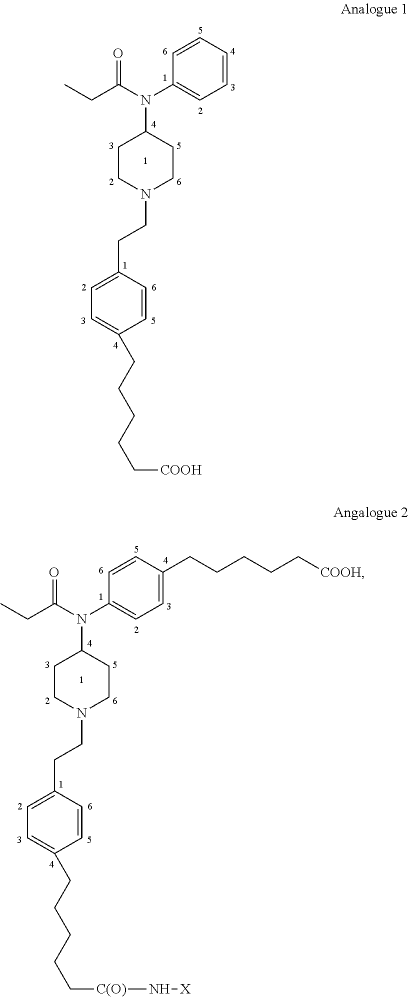

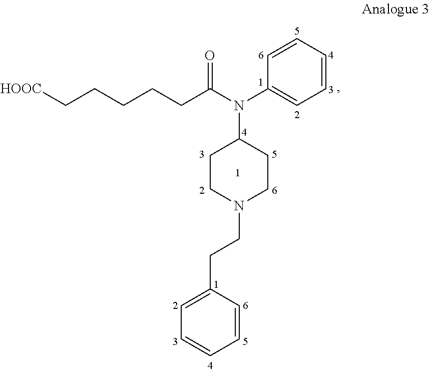 [SMILES: O=C(O)CCCCCC(=O)N(c1ccccc1)C1CCN(CCc2ccccc2)CC1]

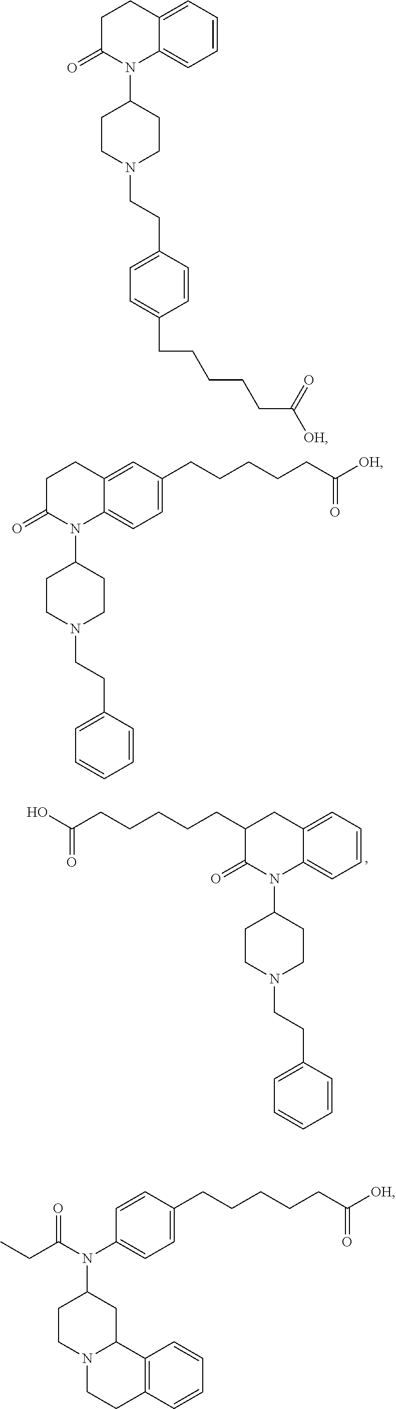

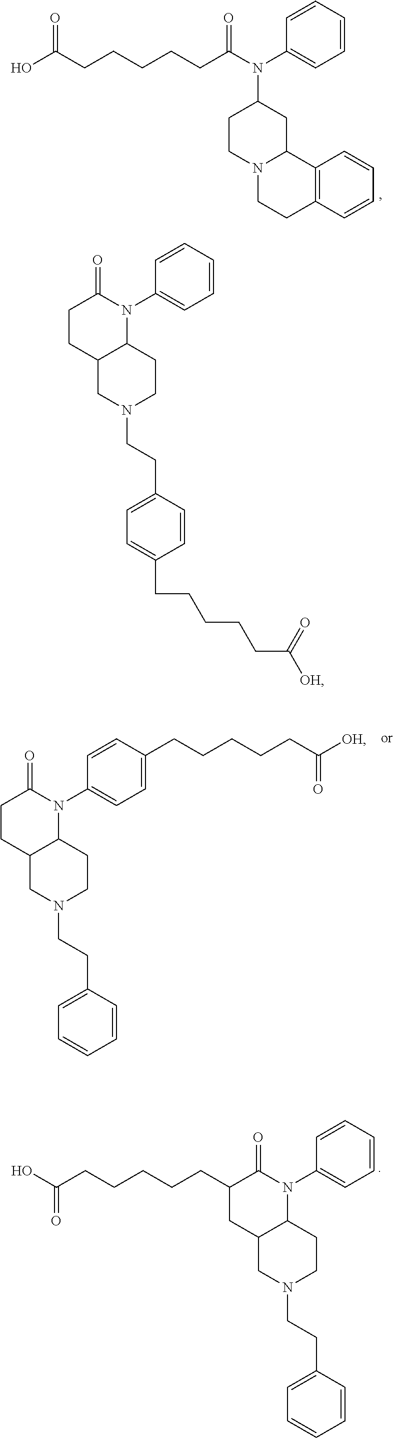 [SMILES: O=C(O)CCCCCC(=O)N(c1ccccc1)C1CCN2CCc3ccccc3C2C1 ; O=C(O)CCCCCc1ccc(CCN2CCC3C(CCC(=O)N3c3ccccc3)C2)cc1 ; O=C(O)CCCCCc1ccc(N2C(=O)CCC3CN(CCc4ccccc4)CCC32)cc1 ; O=C(O)CCCCCC1CC2CN(CCc3ccccc3)CCC2N(c2ccccc2)C1=O]

5. A conjugate comprising the fentanyl analogue of claim 1 or carfentanil conjugated to an adenoviral capsid protein, optionally a hexon protein, a penton base protein, or a fiber protein.

6. A conjugate comprising the fentanyl analogue of claim 2 conjugated to an adenoviral capsid protein, optionally a hexon protein, a penton base protein, or a fiber protein.

7. A conjugate comprising the fentanyl analogue of claim 3 conjugated to an adenoviral capsid protein, optionally a hexon protein, a penton base protein, or a fiber protein.

8. A conjugate comprising the fentanyl analogue of claim 4 conjugated to an adenoviral capsid protein, optionally a hexon protein, a penton base protein, or a fiber protein.

9. The conjugate of claim 5 having the following structure of any of Formulae 1a-7a: 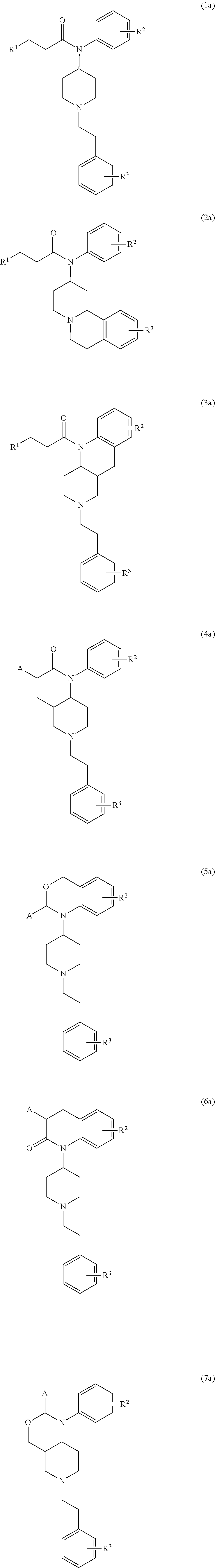
wherein
A is H or -alkyl-R¹;
R¹, R², and R³ are each independently H, —(C₁-C₁₂ alkyl)-COOH, or —(C₁-C₁₂ alkyl)-C(O)NH—X, provided that at least one of R¹, R², and R³ is —(C₁-C₁₂ alkyl)-C(O)NH—X, and
X is an adenoviral capsid protein;
or having the structure of any one of Formulae 8a-10a: 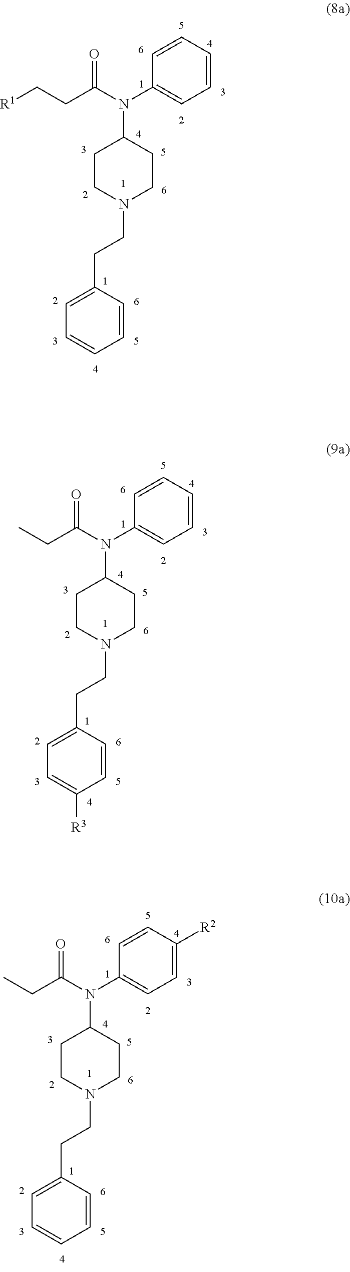
wherein R¹, R², and R³ are each —(C₁-C₁₂ alkyl)-C(O)NH—X, and X is an adenoviral capsid protein;
or having the structure: 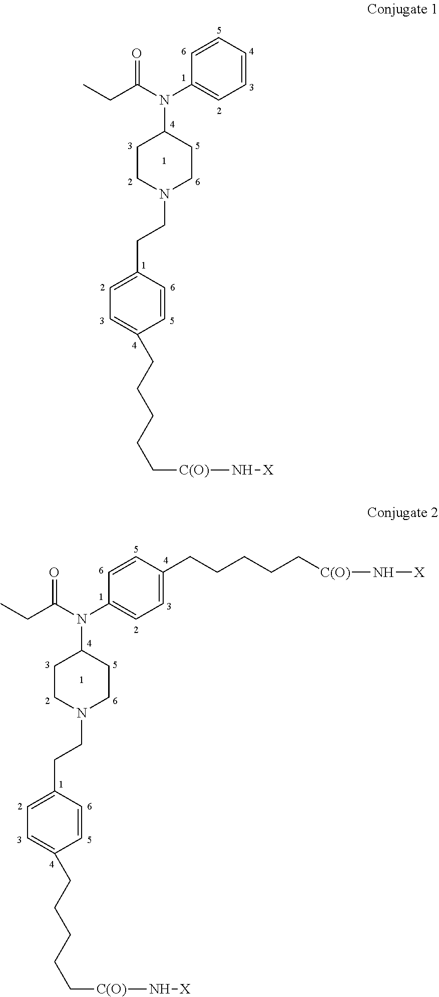 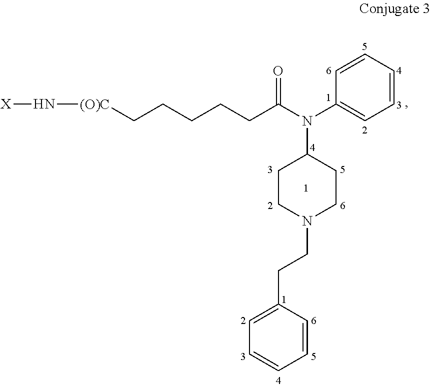 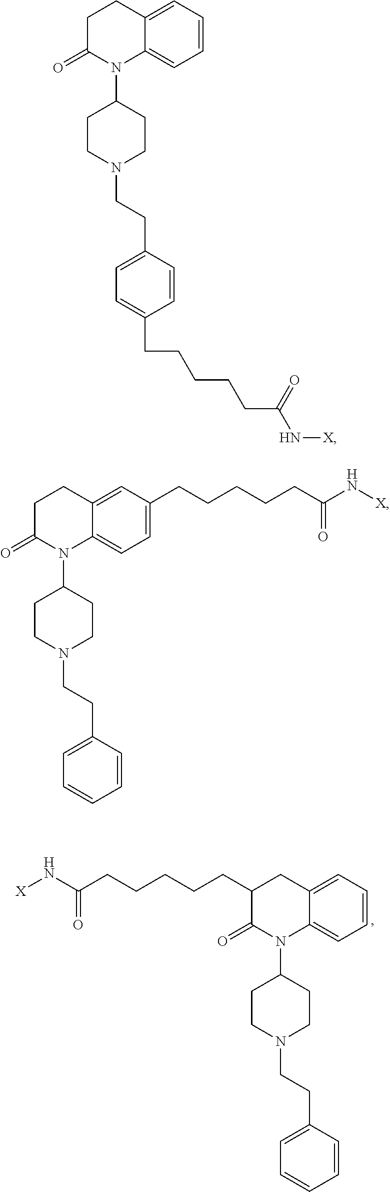 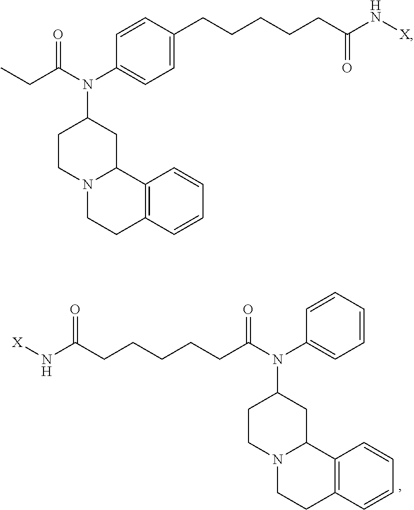 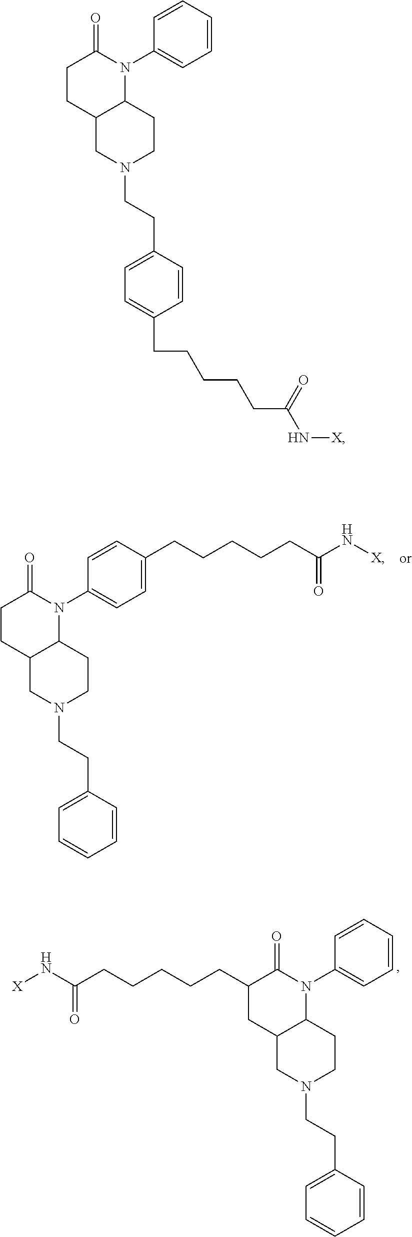
wherein X is an adenoviral capsid protein.

10. (canceled)

11. The conjugate of claim 5, wherein the conjugate comprises carfentanil and has the formula: 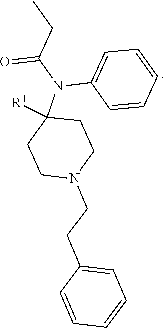
wherein R¹ is —(C₁-C₁₂ alkyl)-C(O)NH—X, and X is an adenoviral capsid protein.

12. The conjugate of claim 5, wherein the adenovirus capsid protein is isolated or purified.

13. (canceled)

14. The conjugate of claim 5, wherein the adenovirus capsid protein is synthetic or recombinant.

15. The conjugate of claim 5, wherein the conjugate comprises a disrupted adenovirus, or one or more additional adenovirus capsid proteins.

16. The conjugate of claim 5, wherein the adenovirus is a human adenovirus, optionally a serotype 5 adenovirus.

17. The conjugate of claim 5, wherein the conjugate comprises an adenovirus disrupted by exposure to heat and/or detergents.

18. A composition comprising the fentanyl analogue of claim 1 or conjugate comprising same and a pharmaceutically acceptable carrier.

19. A method of inducing an immune response against fentanyl in a subject, and/or prevent or treat fentanyl addiction and/or reduce the likelihood of overdose in a subject, optionally a human, which method comprises administering to the subject the conjugate of claim 5.

20. (canceled)

21. (canceled)

22. (canceled)

23. A method of preparing a conjugate comprising the fentanyl analogue of claim 1 or carfentinil and an adenovirus capsid protein, the method comprising combining the fentanyl analogue with an adenovirus capsid protein, whereby the fentanyl analogue is conjugated to the capsid protein.

24. The method of claim 23, wherein the fentanyl analogue and the capsid protein are combined in the presence of N-hydroxysulfosuccinimide (sulfo-NHS) and/or 1-ethyl-3-[3-dimethylaminopropyl]carbodiimide hydrochloride (EDC).

25. The method of claim 23, wherein the method comprises combining the fentanyl analogue with a disrupted adenovirus, and the fentanyl analogue conjugates with a capsid protein of the disrupted adenovirus.
